SEQUENCE LISTING (50 sequences)

SEQ ID NO: 1 - protein, 10 aa, synthetic construct
GYIFSNYWIQ

SEQ ID NO: 2 - protein, 17 aa, synthetic construct
EILPGSGSTEYTENFKD

SEQ ID NO: 3 - protein, 13 aa, synthetic construct
YFFGSSPNWYFDV

SEQ ID NO: 4 - protein, 11 aa, synthetic construct
GASENIYGALN

SEQ ID NO: 5 - protein, 7 aa, synthetic construct
GATNLAD

SEQ ID NO: 6 - protein, 9 aa, synthetic construct
QNVLNTPLT

SEQ ID NO: 7 - protein, 122 aa, synthetic construct [sequence omitted]

SEQ ID NO: 8 - protein, 107 aa, synthetic construct [sequence omitted]

SEQ ID NO: 9 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 447 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, synthetic construct [sequence omitted]

SEQ ID NO: 12 - protein, 122 aa, synthetic construct [sequence omitted]

SEQ ID NO: 13 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, synthetic construct
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, synthetic construct
GASENIYHALN

SEQ ID NO: 18 - protein, 17 aa, synthetic construct
EILPGSGHTEYTENFKD

SEQ ID NO: 19 - protein, 10 aa, synthetic construct
GHIFSNYWIQ

SEQ ID NO: 20 - protein, 448 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, synthetic construct
SYAIS

SEQ ID NO: 22 - protein, 17 aa, synthetic construct
GIGPFFGTANYAQKFQG

SEQ ID NO: 23 - protein, 7 aa, synthetic construct
DTPYFDY

SEQ ID NO: 24 - protein, 11 aa, synthetic construct
SGDSIPNYYVY

SEQ ID NO: 25 - protein, 7 aa, synthetic construct
DDSNRPS

SEQ ID NO: 26 - protein, 11 aa, synthetic construct
QSFDSSLNAEV

SEQ ID NO: 27 - protein, 116 aa, synthetic construct
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, synthetic construct
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, synthetic construct
NYIS

SEQ ID NO: 30 - protein, 17 aa, synthetic construct
IIDPDDSYTEYSPSFQG

SEQ ID NO: 31 - protein, 8 aa, synthetic construct
YEYGGFDI

SEQ ID NO: 32 - protein, 11 aa, synthetic construct
SGDNIGNSYVH

SEQ ID NO: 33 - protein, 7 aa, synthetic construct
KDNDRPS

SEQ ID NO: 34 - protein, 9 aa, synthetic construct
GTYDIESYV

SEQ ID NO: 35 - protein, 116 aa, synthetic construct
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, synthetic construct
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, synthetic construct
SSYYVA

SEQ ID NO: 38 - protein, 17 aa, synthetic construct
AIYTGSGATYKASWAKG

SEQ ID NO: 39 - protein, 13 aa, synthetic construct
DGGYDYPTHAMHY

SEQ ID NO: 40 - protein, 11 aa, synthetic construct
QASQNIGSSLA

SEQ ID NO: 41 - protein, 7 aa, synthetic construct
GASKTHS

SEQ ID NO: 42 - protein, 12 aa, synthetic construct
QSTKVGSSYGNH

SEQ ID NO: 43 - protein, 123 aa, synthetic construct
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, synthetic construct
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, synthetic construct
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, synthetic construct
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0347443 A1
(43) Pub. Date: Nov. 5, 2026

(54) SUPPLEMENTAL DOSAGE AND ADMINISTRATION OF ANTI-C5 ANTIBODIES FOR TREATING HEMATOPOIETIC STEM CELL TRANSPLANT-ASSOCIATED THROMBOTIC MICROANGIOPATHY (HSCT-TMA)

(71) Applicant: ALEXION PHARMACEUTICALS, INC., Boston, MA (US)

(72) Inventors: Stephan ORTIZ, Wellesley, MA (US); Jonathan MONTELEONE, Raleigh, NC (US); Xiaofeng Wang REARS, Boston, MA (US)

(21) Appl. No.: 19/108,880

(22) PCT Filed: Sep. 1, 2023
(86) PCT No.: PCT/US2023/031836

§ 371 Date: Mar. 5, 2025

Related U.S. Application Data
(60) Provisional application No. 63/404,061, filed on Sep. 6, 2022.
(60) Provisional application No. 63/440,984, filed on Jan. 25, 2023.

(51) Int. Cl. C07K 16/18; A61K 39/00; A61P 7/02

(52) U.S. Cl. CPC C07K 16/18; A61P 7/02; A61K 2039/505; A61K 2039/545; C07K 2317/24

(57) ABSTRACT

Provided herein are methods for treating HSCT-TMA in a particular patient sub-population (e.g., HSCT-TMA patients with body weight≥30 kg who received a transfusion (e.g., platelet or red blood cell (RBC) transfusion) in the 4 weeks following a maintenance dose), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at particular loading, maintenance, and supplemental dose amounts, and according to a specific schedule).

### CROSS-REFERENCE TO RELATED APPLICATIONS

This application claims priority to, and the benefit of, U.S. Provisional Application No. 63/404,061 (filed Sep. 6, 2022) and U.S. Provisional Application No. 63/440,984 (filed Jan. 25, 2023). The entire contents of the aforementioned applications is incorporated herein by reference.

### BACKGROUND

Thrombotic microangiopathy (TMA) is a post-transplant complication of hematopoietic stem cell transplant (HSCT) that may affect 10% to 35% of HSCT recipients (Dvorak et al., Front Pediatr. 2019; 7:133); Jodele et al., Blood Rev. 2015; 29 (3): 191-204; Seaby et al., Pediatr Nephrol. 2018; 33 (9): 1489-1500). It presents via endothelial injury and affects the kidney and other organs. It is estimated that 30% of patients with HSCT TMA will present with severe disease (Rosenthal et al., J. Blood Med. 2016; 7:181-186), and in these cases, patients with HSCT-TMA develop systemic vascular injury manifesting in kidney damage, serositis, pulmonary hypertension, and multisystem organ failure. Survival of patients with HSCT-TMA at 1 year has been reported to range from 18% to 40% (Wanchoo et al., Am. J. Kidney Dis. 2018; 72 (6): 857-865). Severe HSCT-TMA is associated with long-term morbidity and mortality rates of approximately 80%. Studies have shown that the large majority of patients die within 6 months (Cho et al., Bone Marrow Transplant. 2008; 41 (9): 813-820; Cho et al., Transplantation. 2010; 90 (8): 918-926; Oran, 2007). Another study showed 9% overall survival of patients with severe HSCT-TMA who did not receive TMA targeted therapy, with all mortality occurring within 10 months of the TMA diagnosis (Jodele, et al., Blood. 2014b;124 (4): 645-653).

In pediatric patients, HSCT-TMA typically occurs early post allogeneic HSCT, with a median diagnosis at 35 to 47 days post-HSCT, and 88% to 92% occurring before Day+100. However, cases have been reported up to 2 years post-HSCT. Autologous recipients may develop HSCT TMA even earlier, with a median of 18 days post-HSCT (Dvorak et al., Front Pediatr. 2019; 7:133).

Endothelial injury is fundamental to the pathogenesis of HSCT-TMA, with dysregulated complement activation likely to be a consequence of the endothelial damage. Risk factors associated with HSCT-TMA development that also initiate endothelial damage include calcineurin inhibitors (CNIs), infections, and conditioning regimens (high dose chemotherapy or total body irradiation) (Khosla, et al., Bone Marrow Transplant. 2018; 53 (2): 129-137; Masias, et al., Blood. 2017; 129 (21): 2857-2863).

Currently, there are no approved therapies for the treatment of HSCT-TMA. Primary intervention for HSCT-TMA involves withdrawal of the offending agent (e.g., CNIs) and/or treatment of any trigger conditions (e.g., treatment for infections) (Dvorak, et al., Front Pediatr. 2019; 7:133); Seaby et al., Pediatr. Nephrol. 2018; 33 (9): 1489-1500). In some patients, withdrawal of the offending agent and/or treatment of any associated trigger condition does not reverse the HSCT-TMA. Accordingly, it is an object of the present disclosure to provide improved methods for treating patients with HSCT-TMA.

### SUMMARY

By way of the present invention, it has been discovered that HSCT-TMA patients treated with ravulizumab according to a particular clinical dosage regimen (e.g., at particular loading and maintenance dose amounts and according to a specific dosing schedule) who received one or more transfusions, e.g., platelet or red blood cell (RBC) transfusions, underwent faster clearance of the antibody (e.g., compared to non-transfused patients) and thereby treatment of the transfused cohort of HSCT-TMA patients with a supplemental dose of ravulizumab provided meaningful clinical benefit. Specifically, using a supplemental dosing simulation model, the inventors have identified that delivery of a supplemental dose of ravulizumab to attain a mean (standard deviation) minimum and maximum plasma concentration of ravulizumab during a dosing interval that is between 474 (200) μg/mL and 1350 (430) μg/mL, respectively, provided a more effective treatment to HSCT-TMA patients who have undergone transfusion, e.g., RBC transfusion, during treatment. Particularly, as exemplified in the non-limiting Examples, HSCT patients with body weight≥30 kg who received any (e.g., one, two, three, or more) RBC transfusions in the 4 weeks following a maintenance dose were administered a supplemental dose of ravulizumab 4 weeks after that maintenance dose. The supplemental ravulizumab dose regimen achieved immediate, complete, and sustained terminal complement inhibition. The supplemental dosing scheme was also applied to effectively treat HSCT-TMA patients in the lower body weight (e.g., <30 kg) and particularly ultra-low body weight (e.g., <20 kg, and particularly <10 kg) cohorts.

In other embodiments, as exemplified in the non-limiting Examples, HSCT patients with body weight <30 kg who received any (e.g., one, two, three, or more) RBC transfusions within a specified timeframe (e.g., 2 weeks for patients who are <20 kg and 4 weeks for patients who are ≥20 kg (e.g., 20 kg<30 kg; 30 kg<40 kg; 40 kg<60 kg; 60 kg<100 kg; or ≥100 kg)) following a maintenance dose were administered a supplemental dose of ravulizumab at the specified timeframe after that maintenance dose. The supplemental ravulizumab dose regimen achieved immediate, complete, and sustained terminal complement inhibition.

Accordingly, provided herein are methods for treating HSCT-TMA in a particular patient sub-population (e.g., HSCT-TMA patients with body weight ≥30 kg who received a transfusion (e.g., platelet or RBC transfusion) in the 4 weeks following a maintenance dose), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at particular loading, maintenance, and one or more supplemental dose amounts, and according to a specific schedule). Also provided herein are methods for treating HSCT-TMA in a particular patient sub-population, e.g., HSCT-TMA patients with body weight <30 kg who received a transfusion (e.g., platelet or RBC transfusion) in the 2 weeks (<20 kg) or 4 weeks (>20 kg to 30 kg) following a maintenance dose), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at particular loading, maintenance, and one or more supplemental dose amounts, and according to a specific schedule).

An exemplary anti-C5 antibody is ravulizumab (ULTOMIRIS®) comprising the heavy and light chains having the sequences shown in SEQ ID NOs: 14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2 and CDR3 domains of the heavy chain variable (VH) region of ravulizumab having the sequence shown in SEQ ID NO: 12, and the CDR1, CDR2 and CDR3 domains of the light chain variable (VL) region of ravulizumab having the sequence shown in SEQ ID NO: 8. In another embodiment, the antibody comprises CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively. In another embodiment, the antibody comprises VH and VL regions having the amino acid sequences set forth in SEQ ID NO:12 and SEQ ID NO:8, respectively. In another embodiment, the antibody comprises a heavy chain constant region as set forth in SEQ ID NO: 13.

In another embodiment, the antibody comprises a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the antibody comprises CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention.

In another embodiment, the anti-C5 antibody comprises the heavy and light chain CDRs or variable regions of the BNJ421 antibody (described in WO2015134894 and U.S. Pat. No. 9,079,949). In another embodiment, the anti-C5 antibody comprises the heavy and light chain CDRs or variable regions of the 7086 antibody (see U.S. Pat. Nos. 8,241,628 and 8,883,158). In another embodiment, the anti-C5 antibody comprises the heavy and light chain CDRs or variable regions of the 8110 antibody (see U.S. Pat. Nos. 8,241,628 and 8,883,158). In another embodiment, the anti-C5 antibody comprises the heavy and light chain CDRs or variable regions of the 305LO5 antibody (see U.S. Pat. No. 9,765,135). In another embodiment, the anti-C5 antibody comprises the heavy and light chain CDRs or variable regions of the SKY59 antibody. In another embodiment, the anti-C5 antibody comprises the heavy and light chain CDRs or variable regions of the REGN3918 antibody.

In another embodiment, the anti-C5 antibody is a biosimilar of eculizumab (SOLIRIS®). For example, in one embodiment, the anti-C5 antibody is, for example, ABP 959 antibody (manufactured by Amgen Inc., USA), ELIZARIA® (manufactured by Generium JNC, Russia), SB12 (manufactured by Samsung Bioepis, Incheon, South Korea), ISU305 (eculizumab biosimilar from ISU Abxis, South Korea), ABLYZER (eculizumab biosimilar from CinnaGen, Iran), BCD 148 (eculizumab biosimilar from Biocad Medical, Quebec, Canada), tesidolumab (manufactured by Novartis), Crovalimab (manufactured by Roche), CAN106 (manufactured by CanBridge Pharmaceuticals, China) or Pozelimab (manufactured by Regeneron).

In another embodiment, the antibody competes for binding with, and/or binds to the same epitope on C5 as any of the above-mentioned antibodies. In another embodiment, the antibody has at least about 90% variable region amino acid sequence identity to any of the above-mentioned antibodies (e.g., at least about 90%, 95% or 99% variable region identity with SEQ ID NO: 12 or SEQ ID NO:8).

In another embodiment, the antibody binds to human C5 at pH 7.4 and 25 ° C with an affinity dissociation constant (K_{D}) that is in the range 0.1 nM≤K_{D}≤1 nM. In another embodiment, the antibody binds to human C5 at pH 7.4 and 25 ° C with an affinity dissociation constant (K_{D}) of about 0.5 nM. In another embodiment, the antibody binds to human C5 at pH 6.0 and 25 ° C with a K_{D}≥10 nM. In another embodiment, the antibody binds to human C5 at pH 6.0 and 25 ° C with a K_{D} of about 22 nM. In yet another embodiment, the [(K_{D} of the antibody or antigen-binding fragment thereof for human C5 at pH 6.0 and at 25 ° C)/(K_{D} of the antibody or antigen-binding fragment thereof for human C5 at pH 7.4 and at 25 ° C)] of the antibody is greater than 25.

In certain embodiments, dosage regimens described herein provide an optimal desired response (e.g., an effective treatment). In some embodiments, effective treatment comprises attaining an immediate, complete, and sustained terminal complement inhibition in the patient with HSCT-TMA.

In accordance with the present disclosure, methods for treating HSCT-TMA in a patient who has undergone transfusion (e.g., platelet or RBC transfusion) is provided, wherein the anti-C5 antibody (or antigen-binding fragment) of the disclosure is administered in a dosing cycle comprising phased dosing, followed by maintenance dosing, and supplemental dosing. In embodiments, the anti-C5 antibody, or antigen binding fragment thereof, is administered a loading dose once on Day 1 (e.g., of the administration cycle), at a loading dose once on Day 5 (e.g., of the administration cycle), at a loading dose once on Day 10 (e.g., of the administration cycle) and a maintenance dose every eight weeks thereafter starting at Day 15. In another embodiment, the maintenance dose of the anti-C5 antibody, or antigen binding fragment thereof, is administered every eight weeks after treatment (e.g., an administration cycle) for an extension period up to two years.

In embodiments, the anti-C5 antibody, or antigen binding fragment thereof, is administered at a loading dose once on Day 1 (e.g., of the administration cycle), at a loading dose once on Day 5 (e.g., of the administration cycle), at a loading dose once on Day 10 (e.g., of the administration cycle), a maintenance dose every eight weeks thereafter starting at Day 15, along with a supplemental dose. In an embodiment, a supplemental dose is administered to a patient who receives a red blood cell (RBC) transfusion within four weeks of a maintenance dose, wherein the supplemental dose is administered four weeks after that maintenance dose. In another embodiment, a supplemental dose is administered to a patient weighing ≥30 kg who receives a RBC transfusion within four weeks of a maintenance dose, wherein the supplemental dose is administered four weeks after that maintenance dose at a dose of: 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg. In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives a red blood cell (RBC) transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

In another embodiment, a method of treating a human patient with HSCT-TMA, preferably an RBC-transfused HSCT-TMA patient, is provided, the method comprising administering to the patient an effective amount of an anti-C5 antibody comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention, wherein the anti-C5 antibody is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives a red blood cell (RBC) transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

In another embodiment, a method of treating a human patient with HSCT-TMA, preferably an RBC-transfused HSCT-TMA patient, is provided, the method comprising administering to the patient an effective amount of ravulizumab, wherein ravulizumab is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives a red blood cell (RBC) transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.
         and wherein the treatment results in a reduction in LDH levels to within normal levels or to within 50% below what is considered the ULN level (e.g., within 105-333 IU/L (international units per liter), a serum trough concentration of the anti-C5 antibody, or antigen binding fragment thereof, of at least 175 μg/mL or greater, and/or a free C5 concentration of 0.5 μg/mL or less (e.g., 0.4 μg/mL, 0.3 μg/mL, 0.2 μg/mL, or 0.1 μg/mL or less).

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 300 mg; and
(e) a supplemental dose of 300 mg, to the patient who receives RBC transfusion within a specified timeframe of a maintenance dose, wherein the supplemental dose is administered at the specified timeframe following the maintenance dose, wherein the specified timeframe is two weeks.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 400 mg; and
(e) a supplemental dose of 400 mg, to the patient who receives RBC transfusion within a specified timeframe of a maintenance dose, wherein the supplemental dose is administered at the specified timeframe following the maintenance dose, wherein the specified timeframe is two weeks.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 600 mg, and
(e) a supplemental dose of 600 mg, to the patient who receives RBC transfusion within a specified timeframe of a maintenance dose, wherein the supplemental dose is administered at the specified timeframe following the maintenance dose, wherein the specified timeframe is two weeks.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 800 mg, and
(e) a supplemental dose of 800 mg, to the patient who receives RBC transfusion within a specified timeframe of a maintenance dose, wherein the supplemental dose is administered at the specified timeframe following the maintenance dose, wherein the specified timeframe is two weeks.

In another embodiment, a method of treating a human patient with HSCT-TMA, preferably to RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 20 to <30 kg:
(a) once on Day 1 at a loading dose of 900 mg;
(b) once on Day 5 at a loading dose of 300 mg;
(c) once on Day 10 at a loading dose of 300 mg;
(d) on Day 15 and every eight weeks thereafter at a maintenance dose of 2100 mg; and
(e) a supplemental dose of 2100 mg, to the patient who receives RBC transfusion within a specified timeframe of a maintenance dose, wherein the supplemental dose is administered at the specified timeframe following the maintenance dose, wherein the specified timeframe is four weeks.

In some embodiments, the supplemental dose is administered if warranted per a clinical algorithm as set forth in FIG. 3. In some embodiments, the supplemental dose is administered if warranted per a clinical algorithm as set forth in Table 7.

In another embodiment, a method of treating a human patient with HSCT-TMA, preferably RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg; and
(d) on Day 15 and every eight weeks thereafter at a maintenance dose of 2700 mg, and wherein the patient receives a RBC transfusion within four weeks of a maintenance dose and is then administered a supplemental dose of 2700 mg four weeks after that maintenance dose.

In another embodiment, a method of treating a human patient with HSCT-TMA, preferably RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg; and
(d) on Day 15 and every eight weeks thereafter at a maintenance dose of 2700 mg, wherein the patient receives a RBC transfusion within four weeks of a maintenance dose and is then administered a supplemental dose of 2700 mg four weeks after that maintenance dose, and wherein the treatment results in immediate, complete, and sustained terminal complement inhibition.

In another embodiment, a method of treating a human patient with HSCT-TMA, preferably RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg; and
(d) on Day 15 and every eight weeks thereafter at a maintenance dose of 3000 mg, and wherein the patient receives a RBC transfusion within four weeks of a maintenance dose and is then administered a supplemental dose of 3000 mg four weeks after that maintenance dose.

In another embodiment, a method of treating a human patient with HSCT-TMA, preferably RBC transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg; and
(d) on Day 15 and every eight weeks thereafter at a maintenance dose of 3000 mg, wherein the patient receives a RBC transfusion within four weeks of a maintenance dose and is then administered a supplemental dose of 3000 mg four weeks after that maintenance dose and wherein the treatment results in immediate, complete, and sustained terminal complement inhibition.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg; and
(d) on Day 15 and every eight weeks thereafter at a maintenance dose of 3300 mg., and wherein the patient receives a RBC transfusion within four weeks of a maintenance dose and is then administered a supplemental dose of 3300 mg four weeks after that maintenance dose.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg; and
(d) on Day 15 and every eight weeks thereafter at a maintenance dose of 3300 mg., wherein the patient receives a RBC transfusion within four weeks of a maintenance dose and is then administered a supplemental dose of 3300 mg four weeks after that maintenance dose and wherein the treatment results in immediate, complete, and sustained terminal complement inhibition.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg; and
(d) on Day 15 and every eight weeks thereafter at a maintenance dose of 3600 mg, and wherein the patient receives a RBC transfusion within four weeks of a maintenance dose and is then administered a supplemental dose of 3600 mg four weeks after that maintenance dose.

In another embodiment, a method of treating a human patient with HSCT-TMA, preferably an RBC-transfused HSCT-TMA patient, is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg; and
(d) on Day 15 and every eight weeks thereafter at a maintenance dose of 3600 mg, wherein the patient receives a RBC transfusion within four weeks of a maintenance dose and is then administered a supplemental dose of 3600 mg four weeks after that maintenance dose and wherein the treatment results in immediate, complete, and sustained terminal complement inhibition.

In some embodiments, wherein the patient undergoes an RBC transfusion within the 2 weeks after a first maintenance dose, the patient receives a post-transfusion supplemental dose of the anti-C5 antibody (e.g., ravulizumab) once only at the mid-way point between 2 maintenance doses. Since the maintenance doses are administered every four weeks (Q4W) for certain lower weight cohorts (e.g., <20 kg; see Table 8), the supplemental dose of the anti-C5 antibody (e.g., ravulizumab) is administered after week 2, e.g., 14 days after the maintenance dose.

In some embodiments, wherein the patient undergoes an RBC transfusion within the 4 weeks after a first maintenance dose, the patient receives a post-transfusion supplemental dose of the anti-C5 antibody (e.g., ravulizumab) once only at the mid-way point between 2 maintenance doses. Since the maintenance doses are administered every eight weeks (Q8W), the supplemental dose of the anti-C5 antibody (e.g., ravulizumab) is administered after week 4, e.g., 28 days after the maintenance dose. In such embodiments, if the patient does not undergo any transfusions during the second maintenance dosing period, then no supplemental dose is needed during this maintenance dosing cycle. Likewise, if the patient undergoes RBC transfusion during the second half of the second maintenance dosing period, then no supplemental dosing is needed during this maintenance dosing cycle. Stated another way, no post-transfusion supplemental dose is required if the first RBC transfusion occurs more than 4 weeks after the preceding maintenance dose. In addition, supplemental dosing is not administered if the patient receives a transfusion during the loading dose phase.

In some embodiments of the treatment method(s) of the present disclosure, the supplemental dosing regimen is continued until no transfusions are administered, in the 2-week period for participants with body weight <20 kg or in the 4-week period for participants with body weight ≥20 kg, following a given maintenance dose.

In accordance with the treatment method described herein, a post-transfusion supplemental dose of the anti-C5 antibody, or antigen binding fragment thereof, (e.g., ravulizumab) is administered to the HSCT-TMA patient once, e.g., only 2 weeks (<20 kg patients) or only 4 weeks (>20 kg patients) after the last maintenance dose, if any amount of RBC transfusion is provided to the patient within the 2 weeks or 4 weeks after the last maintenance dose.

In another embodiment, the anti-C5 antibody, or antigen binding fragment thereof, is administered for one or more administration cycles. In one embodiment, the treatment (e.g., administration cycle) is at least 26 weeks, at least 52 weeks, at least 1.5 years, at least 2 years.

In another embodiment, immediate, complete, and sustained terminal complement inhibition is achieved following administration of a supplement dose of the anti-C5 antibody, or antigen binding fragment thereof (e.g., ravulizumab).

In one embodiment, the methods of treatment disclosed herein further comprise administering best supportive care (BSC) measures. BSC measures include, but are not limited to, transfusion support, corticosteroids, dialysis, and antihypertensive medications.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

In another aspect, the treatment regimens described are sufficient to maintain particular serum trough concentrations of the anti-C5 antibody or antigen binding fragment thereof. In one embodiment, for example, the treatment regimen maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700 μg/mL or greater. In one embodiment, the treatment regimen maintains a serum trough concentration of the anti-C5 antibody or antigen binding fragment thereof of 100 μg/mL or greater, 150 μg/mL or greater, 200 μg/mL or greater, 250 μg/mL or greater, 300 μg/mL or greater, 350 μg/mL or greater, 400 μg/mL or greater, or 450 μg/mL or greater. In another embodiment, the treatment maintains a serum trough concentration of the anti-C5 antibody or antigen binding fragment thereof of between 100 μg/mL and 700 μg/mL; preferably between 300 μg/mL and 600 μg/mL. In another embodiment, the treatment maintains a serum trough concentration of the anti-C5 antibody or antigen binding fragment thereof of about 475 μg/mL. In one embodiment, the treatment regimen maintains a serum peak concentration of the anti-C5 antibody or antigen binding fragment thereof of less than about 1800, 1780, 1760, 1740, 1720, 1700, 1680, 1660, 1640, 1620, 1600, 1580, 1560, 1540, 1520, 1500, 1480, 1460, 1440, 1420, 1400, 1380, 1360, 1340, 1320, 1300, 1280, 1260, 1240, 1220, 1200, 1180, 1160, 1140, 1120, 1100, 1080, 1060, 1040, 1020, 1000, 980, 960, 940, 920, or 900 μg/mL, or less. In embodiments, the treatment maintains a peak serum concentration of the anti-C5 antibody or antigen binding fragment thereof of between 900 μg/mL and 1800 μg/mL; preferably between 1050 μg/mL and 1550 μg/mL. In another embodiment, the treatment maintains a peak serum concentration of the anti-C5 antibody or antigen binding fragment thereof of about 1350 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 270 μg, 280 μg, 290 μg, 300 μg, 320 μg, 340 μg, 360 μg, 380 μg, 400 μg, 420 μg, 440 μg, 460 μg, 480 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 ug, 1600 μg, 1650 μg, 1700 μg, 1750 μg, or more, e.g., 1800 μg of antibody per milliliter of the patient's blood.

In embodiments, to obtain an effective response, the anti-C5 antibody (e.g., ravulizumab) is administered to the patient in an amount and with a frequency to maintain a minimum and maximum plasma concentration (±standard deviation) of the anti-C5 antibody (e.g., ravulizumab) during a dosing interval that is preferably between 474 (+200) μg/mL and 1350 (+430) μg/mL, respectively.

In another embodiment, to obtain an effective response, the anti-C5 antibody is administered to the patient in an amount and with a frequency to maintain a minimum free C5 concentration. In one embodiment, for example, the anti-C5 antibody is administered to the patient in an amount and with a frequency to maintain a free C5 concentration of 0.5 μg/mL or less (e.g., 0.4 μg/mL, 0.3 μg/mL, 0.2 μg/mL, or 0.1 μg/mL or less).

The anti-C5 antibodies, or antigen binding fragments thereof, can be administered to a patient by any suitable means. In one embodiment, the antibodies are formulated for intravenous administration.

The efficacy of the treatment methods provided herein can be assessed using any suitable means. In one embodiment, for a HSCT-TMA patient, the treatment produces at least one therapeutic effect selected from the group consisting of: a reduction or cessation in microangiopathic hemolytic anemia, thrombocytopenia, endothelial injury, kidney damage, kidney failure, serositis, pulmonary hypertension, and multisystem organ failure compared to baseline.

In another embodiment, the treatment results in (a) platelet count ≥50,000/mm³ without transfusion support during the prior 7 days, (b) LDH <1.5×ULN, and (c) absence of schistocytes (if there were schistocytes present at baseline).

In another embodiment, the treatment results in (a) platelet count ≥50,000/mm³ without transfusion support during the prior 7 days, (b) LDH <1.5×ULN, (c) absence of schistocytes (if there were schistocytes present at baseline) and (d) at least 50% reduction of proteinuria from baseline. In one embodiment, proteinuria is as protein/creatinine ratio ≥0.5 mg/mg.

In another embodiment, the treatment results in a favorable hematological response.

In another embodiment, the treatment results in normalization of LDH, resolution of need for red cell and platelet transfusions, and disappearance of schistocytes.

In another embodiment, the treatment results in the patient's ability to maintain hemoglobin ≥8 g/dL without transfusion support. In another embodiment, the treatment results in hemoglobin ≥8 g/dL without transfusion support.

In another embodiment, the treatment results in a decrease in LDH, an increase in platelets, and/or an increase in hemoglobin compared to baseline.

In another embodiment, the treatment results in normal levels of serum creatinine compared to baseline.

In another embodiment, the treatment results in an improvement in TMA-associated organ dysfunction in the renal, cardiovascular, pulmonary, CNS, and/or GI systems compared to baseline.

In another embodiment, the treatment results in terminal complement inhibition.

In another embodiment, the treatment produces a reduction in adverse events.

In another embodiment, the treatment produces a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products. In another embodiment, the treatment produces a shift toward normal levels of thrombomodulin (TM) and/or syndecan-1 (SYND1). In another embodiment, the treatment produces a shift toward normal levels of complement factor Ba.

In another embodiment, the treatment produces a change from baseline in quality of life as assessed via a Quality of Life Assessment (e.g., a Quality of Life Inventory (PedsQL) Scale or an EQ-5D-5L questionnaire).

In another embodiment, the treatment results in a reduction of hemolysis as assessed by lactate dehydrogenase (LDH) levels compared to baseline. In one embodiment, patients treated according to the disclosed methods experience reductions in LDH levels to near normal levels or to within 10%, or within 20% above what is considered the normal level (e.g., within 105-333 IU/L (international units per liter). In another embodiment, the patient's LDH levels are normalized throughout maintenance period of treatment. In another embodiment, the treated patient's LDH levels are normalized at least at least 95% of the time while on the maintenance period of treatment. In another embodiment, the treated patient's LDH levels are normalized at least at least 90%, 85% or 80% of the time while on the maintenance period of treatment. In one embodiment, the patient's LDH levels are ≥1.5 fold above the upper limit of normal (LDH≥1.5×ULN) prior to initiating treatment.

In one embodiment, patients treated according to the disclosed methods experience reductions in LDH levels to within normal levels or to within 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or within 50% below what is considered the ULN level (e.g., within 105-333 IU/L (international units per liter). In one embodiment, the patient's LDH levels are ≥1.5 fold above the ULN (LDH≥1.5×ULN) prior to initiating treatment.

In one embodiment, patients treated according to the disclosed methods experience an LDH percent change compared to baseline of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53% 54%, 55%, 56%, 57%, 58%, 59%, or 60%.

Further provided are kits that include a pharmaceutical composition containing an anti-C5 antibody or antigen binding fragment thereof, such as ravulizumab, and a pharmaceutically acceptable carrier, in a therapeutically effective amount adapted for use in the methods described herein, e.g., for effective treatment of an HSCT-TMA patient who has undergone at least one transfusion, e.g., platelet or RBC transfusion. In one embodiment, the kit comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody or antigen binding fragment thereof in the methods described herein. In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 300 mg; and
(e) a supplemental dose of 300 mg, with instructions that the supplemental dose is administered to a patient who receives a red blood cell (RBC) transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 400 mg; and
(e) a supplemental dose of 400 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 600 mg, and
(e) a supplemental dose of 600 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 800 mg, and
(e) a supplemental dose of 800 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 20 to <30 kg:
(a) once on Day 1 at a loading dose of 900 mg;
(b) once on Day 5 at a loading dose of 300 mg;
(c) once on Day 10 at a loading dose of 300 mg;
(d) on Day 15 and every eight weeks thereafter at a maintenance dose of 2100 mg; and
(e) a supplemental dose of 2100 mg, with instructions that the supplemental dose is administered to a patient who receives RBC transfusion within 4 weeks of a maintenance dose, wherein the supplemental dose is administered four weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg;
(d) on Day 15 and every eight weeks thereafter at a maintenance dose of 2700 mg, and (e) a supplemental dose of 2700 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg;
(d) on Day 15 and every eight weeks thereafter at a maintenance dose of 3000 mg, and (e) a supplemental dose of 3000 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg;
(d) on Day 15 and every eight weeks thereafter at a maintenance dose of 3300 mg, and (e) a supplemental dose of 3300 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg;
(d) on Day 15 and every eight weeks thereafter at a maintenance dose of 3600 mg, and (e) a supplemental dose of 3600 mg, with instructions that the supplemental dose is administered to a patient who receives RBC transfusion within 4 weeks of a maintenance dose, wherein the supplemental dose is administered four weeks after that maintenance dose.

In another aspect, an anti-C5 antibody, or antigen-binding fragment thereof, (e.g., ravulizumab (ULTOMIRIS®)), for use in treatment of an HSCT-TMA patient who has undergone RBC transfusion during a first half of a maintenance phase of treatment is provided, wherein the anti-C5 antibody, or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

Further provided herein are uses of anti-C5, antibody or antigen-binding fragment 0 thereof, (e.g., ravulizumab (ULTOMIRIS®) for treatment of an HSCT-TMA patient who has undergone RBC transfusion during a first half of a maintenance phase of treatment, wherein the anti-C5 antibody, or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, two weeks after a maintenance dose,
      b. 600 or 800 mg to a patient weighing 10 to <20 kg, two weeks after a maintenance dose, or
      c. 2100 mg to a patient weighing 20 to <30 kg, four weeks after a maintenance dose, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

### BRIEF DESCRIPTION OF THE DRAWINGS

FIG. 1 is a graph showing the ravulizumab pharmacokinetic (PK) profile for each patient. At Day 5 and Day 10, ravulizumab concentration was maintained above the 175 μg/mL threshold (dashed line).

FIG. 2 is a graph showing free C5 concentration-time profile for each patient (i.e., pharmacodynamic (PD) profile). All patients demonstrated free C5 concentrations below the 0.5 μg/mL threshold (dashed line).

FIG. 3 is a schematic depicting the clinical algorithm for assessing post-RBC supplemental dosing.

### DETAILED DESCRIPTION

### I. DEFINITIONS

As used herein, the term “subject” or “patient” is a human patient (e.g., a patient having hematopoietic stem cell transplant-associated thrombotic microangiopathy (HSCT-TMA)).

As used herein, the term “pediatric” patient is a human patient that has been classified by a physician or caretaker as belonging to a non-adult category and can include, e.g., newborn (both preterm and of term), infants, children, and adolescents. Typically, pediatric patients are patients under 18 years of age (<18 years of age).

As used herein, the term “adult” patient is a human patient that has been classified by a physician or caretaker as such, e.g., one who is not a newborn, infant, child or adolescent, e.g., based on age, developmental status, physiological features, etc. Typically, adult patients are patients who are 18 years of age or older (≥18 years of age).

HSCT-TMA is a multifactorial disorder caused by systemic vascular endothelial injury that can be triggered by several mechanisms during the transplant process. It presents via endothelial injury and affects the kidney and other organs. It is estimated that 30% of patients with HSCT TMA will present with severe disease (Rosenthal et al., J. Blood Med. 2016; 7:181-186), and in these cases, patients with HSCT-TMA develop systemic vascular injury manifesting in kidney damage, serositis, pulmonary hypertension, and multisystem organ failure. In one embodiment, a TMA diagnosis, is based on all of the following criteria occurring simultaneously: (a) de novo thrombocytopenia or platelet transfusion refractoriness, where: de novo thrombocytopenia is defined as new decline in platelet count to ≤50,000/mm³ and transfusion refractoriness is defined as failure of the platelet increment 10-60 minutes post transfusion to exceed the transfusion trigger, or a rise of less than 10×10⁹/L 20-24 hours post-transfusion after at least 2 platelet transfusions, (b) de novo anemia (defined as a new decline in hemoglobin to ≤8 g/dL) or increase in transfusion requirements (defined as the need to administer more frequent transfusions in order to maintain hemoglobin ≥8 g/dL), (c) either one of the following markers of hemolysis: LDH>1.5×ULN or presence of schistocytes ≥2 high power field (HPF) in peripheral blood smear, (d) proteinuria on spot urinalysis where proteinuria is defined as protein/creatinine ratio ≥0.5 mg/mg. The presence of proteinuria should be confirmed by a second measurement (with the 2 measurements at least 4 hours apart); (e) presence of hypertension defined as the presence of any one of the 3 conditions: a systolic pressure ≥139 mm Hg or a diastolic pressure greater than 80 mm Hg on 2 consecutive measurements at least 1 hour apart.

As used herein, “effective treatment” refers to treatment producing a beneficial effect, e.g., amelioration of at least one symptom of a disease or disorder. A beneficial effect can take the form of an improvement over baseline, e.g., an improvement over a measurement or observation made prior to initiation of therapy according to the method. Effective treatment may refer to alleviation of at least one symptom of HSCT-TMA (e.g., microangiopathic hemolytic anemia, thrombocytopenia, endothelial injury, kidney damage, kidney failure, serositis, pulmonary hypertension, and multisystem organ failure).

The term “effective amount” refers to an amount of an agent that provides the desired biological, therapeutic and/or prophylactic result. That result can be reduction, amelioration, palliation, lessening, delaying and/or alleviation of one or more of the signs, symptoms or causes of a disease, or any other desired alteration of a biological system. In one example, an “effective amount” is the amount of anti-C5 antibody, or antigen binding fragment thereof, clinically proven to alleviate at least one symptom of HSCT-TMA (e.g., microangiopathic hemolytic anemia, thrombocytopenia, endothelial injury, kidney damage, kidney failure, serositis, pulmonary hypertension, and multisystem organ failure). An effective amount can be administered in one or more administrations.

As used herein, the term “loading dose” refers to the initial doses administered during an administration cycle (e.g., on Days 1, 5, and 10).

As used herein, the terms “maintenance” and “maintenance phase” are used interchangeably and refer to the second phase of treatment (e.g., doses administered every eight weeks starting at Day 15). In certain embodiments, treatment is continued as long as clinical benefit is observed or until unmanageable toxicity or disease progression occurs.

As used herein, the term “supplemental dose” refers to an additional dose administered to an HSCT-TMA patient (e.g., having a body weight ≥30 kg) who received a RBC transfusion in the 4 weeks following a maintenance dose.

As used herein, the term “blood transfusion” refers to a procedure in which whole blood or parts of blood are put into a patient's bloodstream, e.g., through a vein. The blood may be donated by another person or it may have been taken from the patient and stored until needed.

As used herein, the term “red blood cell (RBC) transfusion” refers to a transfusion of red blood cells, i.e., concentrated erythrocytes obtained from donated whole blood or by donor apheresis, into a patient's bloodstream, e.g., through a vein. The majority of RBC transfusions are by infusion of packed RBCs that are prepared either by removing plasma from whole blood collection or by apheresis, followed by storage with additives to enhance cell viability and storage.

As used herein, the term “serum trough level” refers to the lowest level that the agent (e.g., the anti-C5 antibody, or antigen binding fragment thereof) or medicine is present in the serum. In contrast, a “peak serum level,” refers to the highest level of the agent in the serum. The “average serum level,” refers to the mean level of the agent in the serum over time.

The term “antibody” describes a polypeptide comprising at least one antibody-derived antigen binding site (e.g., VH/VL region or Fv, or CDR). Antibodies include known forms of antibodies, e.g., the antibody can be a human antibody, a humanized antibody, a bispecific antibody or a chimeric antibody. The antibody also can be a Fab, Fab′2, ScFv, SMIP, Affibody®, nanobody or a single-domain antibody. The antibody also can be of any of the following isotypes: IgG1, IgG2, IgG3, IgG4, IgM, IgA1, IgA2, IgAsec, IgD, IgE or combinations thereof. The antibody can be a naturally occurring antibody or an antibody that has been altered by a protein engineering technique (e.g., by mutation, deletion, substitution, conjugation to a non-antibody moiety). An antibody can include, for example, one or more variant amino acids (compared to a naturally occurring antibody) that change a property (e.g., a functional property) of the antibody. Numerous such alterations are known in the art that affect, e.g., half-life, effector function, and/or immune responses to the antibody in a patient. The term antibody also includes artificial or engineered polypeptide constructs that comprise at least one antibody-derived antigen binding site.

### II. Anti-C5 Antibodies

Anti-C5 antibodies described herein bind to complement component C5 (e.g., human C5) and inhibit the cleavage of C5 into fragments C5a and C5b. As described above, such antibodies also have, for example, improved pharmacokinetic properties relative to other anti-C5 antibodies (e.g., eculizumab) used for therapeutic purposes.

Anti-C5 antibodies (or VH/VL domains derived therefrom) suitable for use in the methods described herein can be generated using methods known in the art. Alternatively, art recognized anti-C5 antibodies can be used. Antibodies that compete for binding to C5 with any of these art recognized antibodies or antibodies described herein can also be used.

An exemplary anti-C5 antibody is ravulizumab comprising heavy and light chains having the sequences shown in SEQ ID NOs: 14 and 11, respectively, or antigen binding fragments and variants thereof. Ravulizumab (also known as ULTOMIRIS®, BNJ441 and ALXN1210) is described in WO2015134894 and U.S. Pat. No. 9,079,949, the entire teachings of which are hereby incorporated by reference. The terms ravulizumab, BNJ441, and ALXN1210 may be used interchangeably throughout this document, but all refer to the same antibody. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

The polypeptide sequence of ravulizumab, as accessioned in KEGG DRUG Database (https://www.kegg.jp/entry/D11054), provides that the N-terminal amino acid of variable heavy chain is “X”, but the database does not state what X is. The Chemical Abstracts (CAS) for ravulizumab (CAS 1803171-55-2) also provides that N-terminal X is pyroglutamic acid (designated as “chain 1 pyroglutamic acid-1” in the CAS report). Although this information may seem different from the VH sequence of ravulizumab, e.g., a heavy chain variable region polypeptide comprising the amino acid sequence depicted in SEQ ID NO: 12 and/or a heavy chain polypeptide comprising the amino acid sequence depicted in SEQ ID NO: 14, there is alignment between patented sequences and the drug database/CAS sequence because it was recognized in the art that N-Terminal Q in polypeptide and/or antibody sequence cyclizes during process development to yield drug product conversion to pyroglutamate (Pryo-Q) near 100%, as disclosed in Liu et al. (J Pharm Sci. 2019 October; 108 (10): 3194-3200) https://pubmed.ncbi.nlm.nih.gov/31145921/and Nguyen et al. (Int. J. Mol. Sci. 2017 Jul. 20;18 (7): 1575) https://www.researchgate.net/figure/Cyclization-reactions-of-N-terminal-glutamine-and-glutamate-residues-in-a-polypeptide_fig4_318926365. Additional information is provided in page 7 and Table 4 of Xu et al. (MAbs, 2019 February/Mar;11 (2): 239-264) and the following referenced publications: (1) Yu et al., “Investigation of N-terminal glutamatecyclization of recombinant monoclonal antibody in formulation development”, J. Pharm. Biomed. Anal., 2006, 42, 455-463 and Dick et al., “Determination of the origin of the N-terminal pyro-glutamatevariation in monoclonal antibodies using model peptides”, Biotechnol. Bioeng., 2007, 97, 544-553, the disclosures in which are incorporated by reference in their entirety.

In other embodiments, the antibody comprises the heavy and light chain CDRs or variable regions of ravulizumab. Accordingly, in one embodiment, the antibody comprises the CDR1, CDR2 and CDR3 domains of the VH region of ravulizumab having the sequence set forth in SEQ ID NO: 12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs: 19, 18 and 3, respectively, and light chain CDR1, CDR2 and CDR3 domains having the sequences set forth in SEQ ID NOs: 4, 5 and 6, respectively. In another embodiment, the antibody comprises VH and VL regions having the amino acid sequences set forth in SEQ ID NO: 12 and SEQ ID NO:8, respectively.

Another exemplary anti-C5 antibody is antibody BNJ421 comprising heavy and light chains having the sequences shown in SEQ ID NOs: 20 and 11, respectively, or antigen binding fragments and variants thereof. BNJ421 (also known as ALXN1211) is described in WO2015134894 and U.S. Pat. No. 9,079,949, the entire teachings of which are hereby incorporated by reference.

In other embodiments, the antibody comprises the heavy and light chain CDRs or variable regions of BNJ421. Accordingly, in one embodiment, the antibody comprises the CDR1, CDR2 and CDR3 domains of the VH region of BNJ421 having the sequence set forth in SEQ ID NO: 12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs: 19, 18 and 3, respectively, and light chain CDR1, CDR2 and CDR3 domains having the sequences set forth in SEQ ID NOs: 4, 5 and 6, respectively. In another embodiment, the antibody comprises VH and VL regions having the amino acid sequences set forth in SEQ ID NO: 12 and SEQ ID NO:8, respectively.

The exact boundaries of CDRs are defined differently according to different methods. In some embodiments, the positions of the CDRs or framework regions within a light or heavy chain variable domain are as defined by Kabat et al. [(1991) “Sequences of Proteins of Immunological Interest.” NIH Publication No. 91-3242, U.S. Department of Health and Human Services, Bethesda, MD]. In such cases, the CDRs can be referred to as “Kabat CDRs” (e.g., “Kabat LCDR2” or “Kabat HCDR1”). In some embodiments, the positions of the CDRs of a light or heavy chain variable region are as defined by Chothia et al. (Nature, 342:877-83, 1989). Accordingly, these regions can be referred to as “Chothia CDRs” (e.g., “Chothia LCDR2” or “Chothia HCDR3”). In some embodiments, the positions of the CDRs of the light and heavy chain variable regions can be defined by a Kabat-Chothia combined definition. In such embodiments, these regions can be referred to as “combined Kabat-Chothia CDRs.” Thomas, C. et al. (Mol. Immunol., 33:1389-401, 1996) exemplifies the identification of CDR boundaries according to Kabat and Chothia numbering schemes.

Another exemplary anti-C5 antibody is the 7086 antibody described in U.S. Pat. Nos. 8,241,628 and 8,883,158. In one embodiment, the antibody comprises the heavy and light chain CDRs or variable regions of the 7086 antibody (see U.S. Pat. Nos. 8,241,628 and 8,883,158). In another embodiment, the antibody, or antigen binding fragment thereof, comprises heavy chain CDR1, CDR2 and CDR3 domains having the sequences set forth in SEQ ID NOs: 21, 22 and 23, respectively, and light chain CDR1, CDR2 and CDR3 domains having the sequences set forth in SEQ ID NOs: 24, 25 and 26, respectively. In another embodiment, the antibody, or antigen binding fragment thereof, comprises the VH region of the 7086 antibody having the sequence set forth in SEQ ID NO:27, and the VL region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody comprises the heavy and light chain CDRs or variable regions of the 8110 antibody. In another embodiment, the antibody, or antigen binding fragment thereof, comprises heavy chain CDR1, CDR2 and CDR3 domains having the sequences set forth in SEQ ID NOs: 29, 30 and 31, respectively, and light chain CDR1, CDR2 and CDR3 domains having the sequences set forth in SEQ ID NOs: 32, 33 and 34, respectively. In another embodiment, the antibody comprises the VH region of the 8110 antibody having the sequence set forth in SEQ ID NO:35, and the VL region of the 8110 antibody having the sequence set forth in SEQ ID NO:36.

Another exemplary anti-C5 antibody is the 305LO5 antibody described in U.S. Pat. No. 9,765,135. In one embodiment, the antibody comprises the heavy and light chain CDRs or variable regions of the 305LO5 antibody. In another embodiment, the antibody, or antigen binding fragment thereof, comprises heavy chain CDR1, CDR2 and CDR3 domains having the sequences set forth in SEQ ID NOs: 37, 38 and 39, respectively, and light chain CDR1, CDR2 and CDR3 domains having the sequences set forth in SEQ ID NOs: 40, 41 and 42, respectively. In another embodiment, the antibody comprises the VH region of the 305LO5 antibody having the sequence set forth in SEQ ID NO:43, and the VL region of the 305LO5 antibody having the sequence set forth in SEQ ID NO:44.

Another exemplary anti-C5 antibody is the SKY59 antibody (Fukuzawa, T. et al., Sci. Rep., 7:1080, 2017). In one embodiment, the antibody comprises the heavy and light chain CDRs or variable regions of the SKY59 antibody. In another embodiment, the antibody, or antigen binding fragment thereof, comprises a heavy chain comprising SEQ ID NO:45 and a light chain comprising SEQ ID NO:46.

In some embodiments, the anti-C5 antibody comprises the heavy and light chain variable regions or heavy and light chains of the REGN3918 antibody (see U.S. Pat. No. 10,633,434). In some embodiments, the anti-C5 antibody, or antigen-binding fragment thereof, comprises a heavy chain variable region sequence set forth in SEQ ID NO: 47 and a light chain variable region comprising the sequence set forth in SEQ ID NO: 48. In some embodiments, the anti-C5 antibody, or antigen-binding fragment thereof, comprises a heavy chain sequence set forth in SEQ ID NO: 49 and a light chain sequence set forth in SEQ ID NO: 50.

In another embodiment, the anti-C5 antibody is a biosimilar of eculizumab (SOLIRIS®). For example, in one embodiment, the anti-C5 antibody is, for example, ABP 959 antibody (eculizumab biosimilar manufactured by Amgen Inc., USA), ELIZARIA® (eculizumab biosimilar manufactured by Generium JNC, Russia), SB12 (eculizumab biosimilar manufactured by Samsung Bioepis, Incheon, South Korea), ISU305 (eculizumab biosimilar from ISU Abxis, South Korea), ABLYZER (eculizumab biosimilar from CinnaGen, Iran), BCD 148 (eculizumab biosimilar from Biocad Medical, Quebec, Canada), tesidolumab (manufactured by Novartis), Crovalimab (manufactured by Roche), CAN106 (manufactured by CanBridge Pharmaceuticals, China), or Pozelimab (manufactured by Regeneron).

In some embodiments, an anti-C5 antibody described herein comprises a heavy chain CDR1 comprising, or consisting of, the following amino acid sequence: GHIFSNYWIQ (SEQ ID NO: 19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAE VKKPGASVKV SCKASGHIFS NYWIQWVRQA
PGQGLEWMGE ILPGSGHTEY TENFKDRVTM TRDTSTSTVY
MELSSLRSED TAVYYCARYF FGSSPNWYED VWGQGTLVTV
SS.

In some embodiments, an anti-C5 antibody described herein comprises a light chain variable region comprising the following amino acid sequence:

(SEQ ID NO: 8)
DIQMTQSPSS LSASVGDRVT ITCGASENIY GALNWYQQKP
GKAPKLLIYG ATNLADGVPS RFSGSGSGTD FTLTISSLQP
EDFATYYCQN VLNTPLTFGQ GTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can, for example, comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., WO2015134894 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281:23514-24, 2006); (2) the M428L or T250Q/M428L substitutions (Hinton, P. et al., J. Biol. Chem., 279:6213-6, 2004; Hinton, P. et al., J. Immunol., 176:346-56, 2006); and (3) the N434A or T307/E380A/N434A substitutions (Petkova, S. et al., Int. Immunol., 18:1759-69, 2006). The additional substitution pairings: P257I/Q311I, P257I/N434H and D376V/N434H (Datta-Mannan, A. et al., J. Biol. Chem., 282:1709-17, 2007), the disclosures of each of which are incorporated herein by reference in their entirety.

In some embodiments, the variant constant region has a substitution at EU amino acid posaition 255 for valine. In some embodiments, the variant constant region has a substitution at EU amino acid position 309 for asparagine. In some embodiments, the variant constant region has a substitution at EU amino acid position 312 for isoleucine. In some embodiments, the variant constant region has a substitution at EU amino acid position 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, 9, 8, 7, 6, 5, 4, 3 or 2) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434 of a native human IgG Fc constant region, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine al position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312;lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence set forth in SEQ ID NO:14 and/or a light chain polypeptide comprising the amino acid sequence set forth in SEQ ID NO:11. Alternatively, the anti-C5 antibodies for use in the methods described herein, in some embodiments, comprise a heavy chain polypeptide comprising the amino acid sequence set forth in SEQ ID NO:20 and/or a light chain polypeptide comprising the amino acid sequence set forth in SEQ ID NO: 11.

In one embodiment, the antibody binds to C5 at pH 7.4 and 25 ° C (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In one embodiment, the antibody binds to C5 at pH 7.4 and 25 ° C (and, otherwise, under physiologic conditions) with an affinity dissociation constant (K_{D}) that is about 0.5 nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM. In some embodiments, the antibody binds to C5 at pH 6.0 and 25 ° C (and, otherwise, under physiologic conditions) with a K_{D} that is about 22 nM.

In other embodiments, the [(K_{D} of the antibody for C5 at pH 6.0 at 25 ° C)/(K_{D} of the antibody for C5 at pH 7.4 at 25 C)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000)

Methods for determining whether an antibody binds to a protein antigen and/or the affinity for an antibody to a protein antigen are known in the art. The binding of an antibody to a protein antigen, for example, can be detected and/or quantified using a variety of techniques such as, but not limited to, Western blot, dot blot, surface plasmon resonance (SPR) detection (e.g., BIAcore system; Pharmacia Biosensor AB, Uppsala, Sweden and Piscataway, N.J.), or enzyme-linked immunosorbent assay (ELISA; Benny K. C. Lo (2004) “Antibody Engineering: Methods and Protocols,” Humana Press (ISBN: 1588290921); Johne, B. et al., J. Immunol. Meth., 160:191-8, 1993; Jonsson, U. et al., Ann. Biol. Clin., 51:19-26, 1993; Jönsson, U. et al., Biotechniques, 11:620-7, 1991). In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/kₐ. Such determinations can be measured, for example, at 25 C or 37 C (see the working examples). The kinetics of antibody binding to human C5 can be determined, for example, at pH 8.0, 7.4, 7.0, 6.5 and 6.0 via SPR on a BIAcore 3000 instrument using an anti-Fc capture method to immobilize the antibody.

In one embodiment, the anti-C5 antibody, or antigen binding fragment thereof, blocks the cleavage of C5 into C5a and C5b. Through this blocking effect, for example, the pro-inflammatory effects of C5a and the generation of the C5b-9 membrane attack complex (MAC) at the surface of a cell are inhibited.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods known in the art such as, for example, by a conventional hemolytic assay such as the hemolysis assay (Kabat and Mayer (eds.), “Experimental Immunochemistry, 2^{nd} Edition,” 135-240, Springfield, IL, CC Thomas (1961), pages 135-139), or a conventional variation of that assay such as the chicken erythrocyte hemolysis method (Hillmen, P. et al., N. Engl. J. Med., 350:552-9, 2004). Methods for determining whether a candidate compound inhibits the cleavage of human C5 into forms C5a and C5b are known in the art (Evans, M. et al., Mol. Immunol., 32:1183-95, 1995). The concentration and/or physiologic activity of C5a and C5b in a body fluid can be measured, for example, by methods known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect MAC formation.

Hemolytic assays can be used to determine the inhibitory activity of an anti-C5 antibody, or antigen binding fragment thereof, on complement activation. To determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured. The assay is known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55 or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs: 1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90 or 95) % of the complement inhibitory activity of eculizumab in a hemolytic assay or CH50 eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400 or 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as an antibody described herein. The term “binds to the same epitope” with reference to two or more antibodies means that the antibodies bind to the same segment of amino acid residues, as determined by a given method. Techniques for determining whether antibodies bind to the same epitope on C5 with an antibody described herein include, for example, epitope mapping methods, such as, x-ray analyses of crystals of antigen: antibody complexes, and hydrogen/deuterium exchange mass spectrometry (HDX-MS). Antibodies having the same VH and VL or the same CDR1, CDR2 and CDR3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the antibody that is incubated first with the target). Competing antibodies can bind to, for example, the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies can be obtained by various techniques familiar to those skilled in the art. Briefly, spleen cells from an animal immunized with a desired antigen are immortalized, commonly by fusion with a myeloma cell (Köhler, G. & Milstein, C., Eur. J. Immunol., 6:511-9, 1976)). Methods of immortalization include transformation with Epstein Barr Virus, oncogenes, or retroviruses or other methods known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells (Huse, W. et al., Science, 246:1275-81, 1989).

### III. Compositions

Also provided herein are compositions comprising an anti-C5 antibody or antigen binding fragment thereof. In one embodiment, the composition comprises an anti-C5 antibody comprising the CDR1, CDR2 and CDR3 domains in a heavy chain variable region having the sequence set forth in SEQ ID NO: 12, and the CDR1, CDR2 and CDR3 domains in a light chain variable region having the sequence set forth in SEQ ID NO:8. In another embodiment, the anti-C5 antibody comprises heavy and light chains having the sequences shown in SEQ ID NOs: 14 and 11, respectively. In another embodiment, the anti-C5 antibody comprises heavy and light chains having the sequences shown in SEQ ID NOs: 20 and 11, respectively.

The compositions can be formulated as a pharmaceutical solution, e.g., for administration to a subject for the treatment of HSCT-TMA. The pharmaceutical compositions generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt, sugars, carbohydrates, polyols and/or tonicity modifiers.

The compositions can be formulated according to standard methods. Pharmaceutical formulation is an established art (see, for example, Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X)). In some embodiments, a composition can be formulated, for example, as a buffered solution at a suitable concentration and suitable for storage at 2-8 C (e.g., 4 C). In some embodiments, a composition can be formulated for storage at a temperature below OC (e.g., −20 C or −80 C). In some embodiments, the composition can be formulated for storage for up to 2 years (e.g., 1 month, 2 months, 3 months, 4 months, 5 months, 6 months, 7 months, 8 months, 9 months, 10 months, 11 months, 1 year, 1 1/2 years or 2 years) at 2-8 ° C (e.g., 4 C). Thus, in some embodiments, the compositions described herein are stable in storage for at least 1 year at 2-8 ° C (e.g., 4 C). The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. Compositions containing a composition intended for systemic or local delivery, for example, can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

### IV. Methods

Provided herein are methods for treating HSCT-TMA in a particular patient sub-population (e.g., HSCT-TMA patients, e.g., with body weight <30 kg or with body weight ≥30 kg who received a transfusion (e.g., platelet or RBC transfusion) in the 4 weeks following a maintenance dose), comprising administering to the patient an anti-C5 antibody, or antigen binding fragment thereof, wherein the anti-C5 antibody, or antigen binding fragment thereof, is administered (or is for administration) according to a particular clinical dosage regimen (e.g., at particular loading, maintenance, and one or more supplemental dose amounts, and according to a specific schedule).

In one embodiment, the dose of the anti-C5 antibody, or antigen binding fragment thereof, is based on the weight of the patient. For example, in one embodiment, a 300 mg or 600 mg loading dose of the anti-C5 antibody, or antigen binding fragment thereof, is administered to a patient weighing ≥5 to <10 kg. In another embodiment, a 400 mg maintenance dose of the anti-C5 antibody, or antigen binding fragment thereof, is administered to a patient weighing ≥5 to <10 kg. In another embodiment, a 400 mg supplemental dose of the anti-C5 antibody, or antigen binding fragment thereof, is administered to a patient weighing ≥5 to <10 kg. In some embodiments, a 300 mg or 600 mg loading dose of the anti-C5 antibody, or antigen binding fragment thereof, is administered to a patient weighing ≥10 to <20 kg. In another embodiment, a 800 mg maintenance dose of the anti-C5 antibody, or antigen binding fragment thereof, is administered to a patient weighing ≥10 to <20 kg. In another embodiment, a 800 mg supplemental dose of the anti-C5 antibody, or antigen binding fragment thereof, is administered to a patient weighing ≥10 to <20 kg. In some embodiments, a 300 mg or 900 mg loading dose of the anti-C5 antibody, or antigen binding fragment thereof, is administered to a patient weighing ≥20 to <30 kg. In another embodiment, a 2100 mg maintenance dose of the anti-C5 antibody, or antigen binding fragment thereof, is administered to a patient weighing ≥20 to <30 kg. In another embodiment, a 2100 mg supplemental dose of the anti-C5 antibody, or antigen binding fragment thereof, is administered to a patient weighing ≥20 to <30 kg.

For example, in one embodiment, a 300 mg or 1200 mg loading dose of the anti-C5 antibody, or antigen binding fragment thereof, is administered to a patient weighing ≥30 to <40 kg. In another embodiment, a 2700 mg maintenance dose of the anti-C5 antibody, or antigen binding fragment thereof, is administered to a patient weighing ≥30 to <40 kg. In another embodiment, a 2700 mg supplemental dose of the anti-C5 antibody, or antigen binding fragment thereof, is administered to a patient weighing ≥30 to <40 kg.

In another embodiment, a 600 mg or 2400 mg loading dose of the anti-C5 antibody, or antigen binding fragment thereof, is administered to a patient weighing ≥40 to <60 kg. In another embodiment, a 3000 mg maintenance dose of the anti-C5 antibody, or antigen binding fragment thereof, is administered to a patient weighing ≥40 to <60 kg. In another embodiment, a 3000 mg supplemental dose of the anti-C5 antibody, or antigen binding fragment thereof, is administered to a patient weighing ≥40 to <60 kg.

In another embodiment, a 900 mg or 2700 mg loading dose of the anti-C5 antibody, or antigen binding fragment thereof, is administered to a patient weighing ≥60 to <100 kg. In another embodiment, a 3300 mg maintenance dose of the anti-C5 antibody, or antigen binding fragment thereof, is administered to a patient weighing ≥60 to <100 kg. In another embodiment, a 3300 mg supplemental dose of the anti-C5 antibody, or antigen binding fragment thereof, is administered to a patient weighing ≥60 to <100 kg.

In another embodiment, a 900 mg or 3000 mg loading dose of the anti-C5 antibody, or antigen binding fragment thereof, is administered to a patient weighing ≥100 kg. In another embodiment, a 3600 mg maintenance dose of the anti-C5 antibody, or antigen binding fragment thereof, is administered to a patient weighing ≥100 kg. In another embodiment, a 3600 mg supplemental dose of the anti-C5 antibody, or antigen binding fragment thereof, is administered to a patient weighing ≥100 kg.

In certain embodiments, dosage regimens are adjusted to provide the optimum desired response (e.g., an effective response).

In some embodiments, wherein the patient undergoes an RBC transfusion within the 4 weeks after a first maintenance dose, the patient receives a post-transfusion supplemental dose of the anti-C5 antibody (e.g., ravulizumab) once only at the mid-way point between 2 maintenance doses. Since the maintenance doses are administered every four weeks (Q4W) for lower (e.g., <20 kg) weight class and every eight weeks (Q8W) for higher (e.g., ≥20 kg) weight class, the supplemental dose of the anti-C5 antibody (e.g., ravulizumab) is administered after week 2 (<20 kg patients) and after week 4 (≥20 kg patients), e.g., 14 days or 28 days after the maintenance dose. In such embodiments, if the patient does not undergo any transfusions during the second maintenance dosing period, then no supplemental dose is needed during this maintenance dosing cycle. Likewise, if the patient undergoes RBC transfusion during the second half of the second maintenance dosing period, then no supplemental dosing is needed during this maintenance dosing cycle. Stated another way, no post-transfusion supplemental dose is required if the first RBC transfusion occurs more than 4 weeks after the preceding maintenance dose. In addition, supplemental dosing is not administered if the patient receives a transfusion during the loading dose phase.

In accordance with the treatment method described herein, a post-transfusion supplemental dose of the anti-C5 antibody, or antigen binding fragment thereof, (e.g., ravulizumab) is administered to the HSCT-TMA patient once only 4 weeks after the last maintenance dose, if any amount of RBC transfusion is provided to the patient within the 4 weeks after the last maintenance dose.

In another embodiment, the anti-C5 antibody, or antigen binding fragment thereof, is administered for one or more administration cycles. In one embodiment, the treatment (e.g., administration cycle) is 26 weeks. In one embodiment, the anti-C5 antibody, or antigen binding fragment thereof, is administered at a loading dose once on Day 1 (e.g., of the administration cycle), once on Day 5 (e.g., of the administration cycle), once on Day 10 (e.g., of the administration cycle). In another embodiment, the anti-C5 antibody, or antigen binding fragment thereof, is administered is administered a loading dose once on Day 1 (e.g., of the administration cycle), at a loading dose once on Day 5 (e.g., of the administration cycle), at a loading dose once on Day 10 (e.g., of the administration cycle) and a maintenance dose every eight weeks thereafter starting at Day 15. In another embodiment, the anti-C5 antibody, or antigen binding fragment thereof, is administered every eight weeks after treatment (e.g., an administration cycle) for an extension period up to two years (e.g., at a dose of 300 mg, 600 mg, 900 mg, 1200 mg, 2100 mg, 2400 mg, 2700 mg, 3000 mg, 3300 mg or 3600 mg).

In another embodiment, a supplemental dose is administered to a patient who receives a transfusion (e.g., platelet or RBC transfusion) within two weeks or four weeks of a maintenance dose, wherein the supplemental dose is administered two weeks or four weeks after that maintenance dose. In embodiments, a supplemental dose is administered to a patient weighing <20 kg who receives a RBC transfusion within two weeks of a maintenance dose, wherein the supplemental dose is administered two weeks after that maintenance dose at a dose of: 400 mg to a patient weighing ≥5 to <10 kg, 800 mg to a patient weighing ≥10 to <20 kg. In embodiments, a supplemental dose is administered to a patient weighing ≥20<30 kg who receives a RBC transfusion within four weeks of a maintenance dose, wherein the supplemental dose is administered four weeks after that maintenance dose at a dose of: 2100 mg to a patient weighing ≥20<30 kg.

In another embodiment, a supplemental dose is administered to a patient weighing ≥30 kg who receives a RBC transfusion within four weeks of a maintenance dose, wherein the supplemental dose is administered four weeks after that maintenance dose at a dose of: 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg.

In one embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

In another embodiment, a method of treating a human patient with HSCT-TMA is provided, the method comprising administering to the patient an effective amount of an anti-C5 antibody comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively and a variant human Fc constant region that binds to human neonatal Fc receptor (FcRn), wherein the variant human Fc CH3 constant region comprises Met429Leu and Asn435Ser substitutions at residues corresponding to methionine 428 and asparagine 434 of a native human IgG Fc constant region, each according to the EU numbering convention, wherein the anti-C5 antibody is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

In another embodiment, a method of treating a human patient with HSCT-TMA is provided, the method comprising administering to the patient an effective amount of ravulizumab, wherein ravulizumab is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg.

In another embodiment, a method of treating a human patient with HSCT-TMA is provided, the method comprising administering to the patient an effective amount of ravulizumab, wherein ravulizumab is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
   (i) every four weeks thereafter at a maintenance dose of: 300 or 400 mg to a patient weighing 5 to <10 kg or 600 or 800 mg to a patient weighing 10 to <20 kg, or
   (ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
   (i) to a patient weighing <30 kg who receives RBC transfusion within a specified timeframe of a maintenance dose, wherein the supplemental dose is administered at the specified timeframe following the maintenance dose, at a dose of:
      a. 300 or 400 mg to a patient weighing 5 to <10 kg, wherein the specified timeframe is two weeks,
      b. 600 or 800 mg to a patient weighing 10 to <20 kg, wherein the specified timeframe is two weeks, or
      c. 2100 mg to a patient weighing 20 to <30 kg, wherein the specified timeframe is four weeks, or
   (ii) to a patient weighing ≥30 kg who receives a red blood cell (RBC) transfusion within four weeks of a maintenance dose, wherein the supplemental dose is administered four weeks after that maintenance dose at a dose of:
      a. 2700 mg to a patient weighing ≥30 to <40 kg,
      b. 3000 mg to a patient weighing ≥40 to <60 kg,
      c. 3300 mg to a patient weighing ≥60 to <100 kg, or
      d. 3600 mg to a patient weighing ≥100 kg. and

wherein the treatment results in a reduction in LDH levels to within normal levels or to within 50% below what is considered the ULN level (e.g., within 105-333 IU/L (international units per liter), a serum trough concentration of the anti-C5 antibody, or antigen binding fragment thereof, of at least 175 μg/mL or greater, and/or a free C5 concentration of 0.5 μg/mL or less (e.g., 0.4 μg/mL, 0.3 μg/mL, 0.2 μg/mL, or 0.1 μg/mL or less).

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 300 mg; and
(e) a supplemental dose of 300 mg, to a patient who receives RBC transfusion within two weeks of a maintenance dose, wherein the supplemental dose is administered two weeks after the maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 400 mg; and
(e) a supplemental dose of 400 mg, to a patient who receives RBC transfusion within two weeks of a maintenance dose and wherein the supplemental dose is administered two weeks after the maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 10 to <20 kg:
(a) once on Day 1 at a loading dose of 600 mg;
(b) once on Day 5 at a loading dose of 300 mg;
15 (c) once on Day 10 at a loading dose of 300 mg;
(d) on Day 15 and every four weeks thereafter at a maintenance dose of 600 mg, and
(e) a supplemental dose of 600 mg, to a patient who receives RBC transfusion within two weeks of a maintenance dose and wherein the supplemental dose is administered two weeks after the maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 800 mg, and
(e) a supplemental dose of 800 mg, to a patient who receives RBC transfusion within two weeks of a maintenance dose and wherein the supplemental dose is administered two weeks after the maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing 20 to <30 kg:
(a) once on Day 1 at a loading dose of 900 mg;
(b) once on Day 5 at a loading dose of 300 mg;
(c) once on Day 10 at a loading dose of 300 mg;
(d) on Day 15 and every eight weeks thereafter at a maintenance dose of 2100 mg; and
(e) a supplemental dose of 2100 mg, to a patient who receives RBC transfusion within two weeks of a maintenance dose and wherein the supplemental dose is administered four weeks after the maintenance dose.

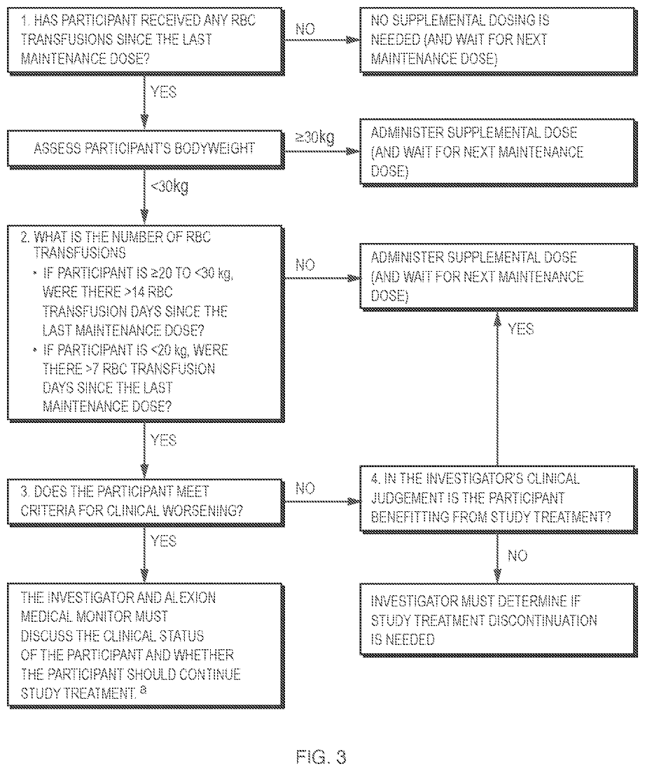

In some embodiments, the supplemental dose is administered if warranted per a clinical algorithm as set forth in FIG. 3. In some embodiments, the supplemental dose is administered if warranted per a clinical algorithm as set forth in Table 7.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg; and
(d) on Day 15 and every eight weeks thereafter at a maintenance dose of 2700 mg, and wherein the patient receives RBC transfusion within four weeks of a maintenance dose and is then administered a supplemental dose of 2700 mg four weeks after that maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody, or antigen binding fragment thereof (e.g., ravulizumab), is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg; and
(d) on Day 15 and every eight weeks thereafter at a maintenance dose of 3000 mg, and wherein the patient receives a RBC transfusion within four weeks of a maintenance dose and is then administered a supplemental dose of 3000 mg four weeks after that maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg; and
(d) on Day 15 and every eight weeks thereafter at a maintenance dose of 3300 mg., and wherein the patient receives a RBC transfusion within four weeks of a maintenance dose and is then administered a supplemental dose of 3300 mg four weeks after that maintenance dose.

In another embodiment, a method of treating a human patient with HSCT-TMA is provided, the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof (e.g., ravulizumab), is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg; and
(d) on Day 15 and every eight weeks thereafter at a maintenance dose of 3600 mg, and wherein the patient receives a RBC transfusion within four weeks of a maintenance dose and is then administered a supplemental dose of 3600 mg four weeks after that maintenance dose.

In another embodiment, immediate, complete, and sustained terminal complement inhibition is achieved following administration of a supplement dose of the anti-C5 antibody, or antigen binding fragment thereof (e.g., ravulizumab).

In one embodiment, the methods of treatment disclosed herein further comprise administering best supportive care (BSC) measures. BSC measures include, but are not limited to, transfusion support, corticosteroids, dialysis, and antihypertensive medications.

In one embodiment, the patient has not previously been treated with eculizumab. In another embodiment, the patient has previously been treated with eculizumab. In another embodiment, the patient has previously been treated with eculizumab and Day 1 (e.g., of the administration cycle) is two weeks or more from the patient's last dose of eculizumab.

### V. Outcomes

Provided herein are methods for treating HSCT-TMA in a patient comprising administering to the patient an anti-C5 antibody. Symptoms of HSCT-TMA include, but are not limited to, e.g., microangiopathic hemolytic anemia, thrombocytopenia, endothelial injury, kidney damage, kidney failure, serositis, pulmonary hypertension, and multisystem organ failure.

Patients treated according to the methods disclosed herein experience improvement in at least one sign of HSCT-TMA. The treatment may produce at least one therapeutic effect selected from the group consisting of, for example, a reduction or cessation in microangiopathic hemolytic anemia, thrombocytopenia, endothelial injury, kidney damage, kidney failure, serositis, pulmonary hypertension, and multisystem organ failure compared to baseline.

In another embodiment, the treatment results in (a) platelet count ≥50,000/mm³ without transfusion support during the prior 7 days, (b) LDH <1.5×ULN, and (c) absence of schistocytes (if there were schistocytes present at baseline).

In another embodiment, the treatment results in (a) platelet count ≥50,000/mm³ without transfusion support during the prior 7 days, (b) LDH <1.5×ULN, (c) absence of schistocytes (if there were schistocytes present at baseline) and (d) at least 50% reduction of proteinuria from baseline. In one embodiment, proteinuria is as protein/creatinine ratio ≥0.5 mg/mg.

In another embodiment, the treatment results in a favorable hematological response.

In another embodiment, the treatment results in normalization of LDH, resolution of need for red cell and platelet transfusions, and disappearance of schistocytes.

In another embodiment, the treatment results in the patient's ability to maintain hemoglobin ≥8 g/dL without transfusion support. In another embodiment, the treatment results in hemoglobin ≥8 g/dL without transfusion support.

In another embodiment, the treatment results in a decrease in LDH, an increase in platelets, and/or an increase in hemoglobin compared to baseline.

In another embodiment, the treatment results in normal levels of serum creatinine compared to baseline.

In another embodiment, the treatment results in an improvement in TMA-associated organ dysfunction in the renal, cardiovascular, pulmonary, CNS, and/or GI systems compared to baseline.

In another embodiment, the treatment results in terminal complement inhibition.

In another embodiment, the treatment produces a reduction in adverse events.

In another embodiment, the treatment produces a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products. In another embodiment, the treatment produces a shift toward normal levels of thrombomodulin (TM) and/or syndecan-1 (SYND1). In another embodiment, the treatment produces a shift toward normal levels of complement factor Ba.

In another embodiment, the treatment produces a change from baseline in quality of life as assessed via a Quality of Life Assessment (e.g., a Quality of Life Inventory (PedsQL) Scale or an EQ-5D-5L questionnaire). The PedsQL 4.0 Generic Core Scales are multidimensional child self-reports and parent proxy-report standardized instruments to measure health related quality of life (QoL) in children and adolescents 2-18 years of age.

In another embodiment, lactate dehydrogenase (LDH) levels can be used to evaluate responsiveness to a therapy. LDH is a marker of intravascular hemolysis (Hill, A. et al., Br. J. Haematol., 149:414-25, 2010; Hillmen, P. et al., N. Engl. J. Med., 350:552-9, 2004; Parker, C. et al., Blood, 106:3699-709, 2005). Red blood cells contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93 (s1): 359 Abs.0903, 2008; Kanakura, Y. et al., Int. J. Hematol., 93:36-46, 2011). LDH concentration obtained at baseline and then serially throughout a treatment period, is an important measure of hemolysis. The normal LDH value range is 105-333 IU/L (international units per liter).

LDH levels can be measured using any suitable test or assay, such as those described by Ferri FF, ed. Ferri's Clinical Advisor 2014. Philadelphia: Pa: Elsevier Mosby; 2014: Section IV-Laboratory tests and interpretation of results. LDH concentration can be measured in various samples obtained from a patient, in particular, serum samples. As used herein, the term “sample” refers to biological material from a subject. Although serum LDH concentration is of interest, samples can be derived from other sources, including, for example, single cells, multiple cells, tissues, tumors, biological fluids, biological molecules or supernatants or extracts of any of the foregoing. Examples include tissue removed for biopsy, tissue removed during resection, blood, urine, lymph tissue, lymph fluid, cerebrospinal fluid, mucous and stool samples. The sample used can vary based on the assay format, the detection method and the nature of the tumors, tissues, cells or extracts to be assayed. Methods for preparing samples are known in the art and can be readily adapted to obtain a sample that is compatible with the method utilized.

In one embodiment, patients treated according to the disclosed methods experience reductions in LDH levels to normal levels or to within 10%, or within 20% above what is considered the normal level (e.g., within 105-333 IU/L). For example, patients treated according to the disclosed methods experience reductions in LDH levels to within normal levels or to within 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or within 50% below what is considered the ULN level (e.g., within 105-333 IU/L (international units per liter). In one embodiment, the patient's LDH levels are ≥1.5 fold above the ULN (LDH≥1.5×ULN) prior to initiating treatment.

In one embodiment, patients treated according to the disclosed methods experience an LDH percent change compared to baseline of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53% 54%, 55%, 56%, 57%, 58%, 59%, or 60%.

In another aspect, the treatment regimens described are sufficient to maintain particular serum trough concentrations of the anti-C5 antibody or antigen binding fragment thereof. In one embodiment, for example, the treatment regimen maintains a serum trough concentration of the anti-C5 antibody or antigen binding fragment thereof of 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 240, 245, 250, 255, 260, 265, 270, 280, 290, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700 μg/mL or greater. In one embodiment, the treatment regimen maintains a serum trough concentration of the anti-C5 antibody or antigen binding fragment thereof of 100 μg/mL or greater, 150 μg/mL or greater, 200 μg/mL or greater, 250 μg/mL or greater, 300 μg/mL or greater, 350 μg/mL or greater, 400 μg/mL or greater, or 450 μg/mL or greater. In another embodiment, the treatment maintains a serum trough concentration of the anti-C5 antibody or antigen binding fragment thereof of between 100 μg/mL and 700 μg/mL; preferably between 300 μg/mL and 600 μg/mL. In another embodiment, the treatment maintains a serum trough concentration of the anti-C5 antibody or antigen binding fragment thereof of about 475 μg/mL. In one embodiment, the treatment regimen maintains a serum peak concentration of the anti-C5 antibody or antigen binding fragment thereof of less than about 1800, 1780, 1760, 1740, 1720, 1700, 1680, 1660, 1640, 1620, 1600, 1580, 1560, 1540, 1520, 1500, 1480, 1460, 1440, 1420, 1400, 1380, 1360, 1340, 1320, 1300, 1280, 1260, 1240, 1220, 1200, 1180, 1160, 1140, 1120, 1100, 1080, 1060, 1040, 1020, 1000, 980, 960, 940, 920, or 900 μg/mL, or less. In embodiments, the treatment maintains a peak serum concentration of the anti-C5 antibody or antigen binding fragment thereof of between 900 μg/mL and 1800 μg/mL; preferably between 1050 μg/mL and 1550 μg/mL. In another embodiment, the treatment maintains a peak serum concentration of the anti-C5 antibody or antigen binding fragment thereof of about 1350 μg/mL.

In another embodiment, to obtain an effective response, the anti-C5 antibody is administered to the patient in an amount and with a frequency to maintain at least 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225μ, 230μ, 235μ, 240μ, 245μ, 250μ, 255μ, 260μ, 270μ, 280μ, 290μ g, 300 μg, 320 μg, 340 μg, 360 μg, 380 μg, 400 μg, 420 μg, 440 μg, 460 μg, 480 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 ug, 1600 μg, 1650 μg, 1700 μg, 1750 μg, or more, e.g., 1800 μg of antibody per milliliter of the patient's blood.

In embodiments, to obtain an effective response, the anti-C5 antibody (e.g., ravulizumab) is administered to the patient in an amount and with a frequency to maintain a minimum and maximum plasma concentration (±standard deviation) of the anti-C5 antibody (e.g., ravulizumab) during a dosing interval that is preferably between 474 (+200) μg/mL and 1350 (+430) μg/mL, respectively.

In one embodiment, patients treated according to the disclosed methods have a free C5 concentration of 0.5 μg/mL or less (e.g., 0.4 μg/mL, 0.3 μg/mL, 0.2 μg/mL, or 0.1 μg/mL or less).

In another embodiment, patients treated according to the disclosed methods achieve an immediate, complete, and sustained terminal complement inhibition.

### VI. Kits and Unit Dosage Forms

Also provided herein are kits that include a pharmaceutical composition containing an anti-C5 antibody or antigen binding fragment thereof, such as ravulizumab, and a pharmaceutically acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having HSCT-TMA. The kit also can include a syringe.

Optionally, the kits include multiple packages of the single-dose pharmaceutical compositions each containing an effective amount of the anti-C5 antibody, or antigen binding fragment thereof, for a single administration in accordance with the methods provided above. Instruments or devices necessary for administering the pharmaceutical composition(s) also may be included in the kits. For instance, a kit may provide one or more pre-filled syringes containing an amount of the anti-C5 antibody or antigen binding fragment thereof.

In one embodiment, a kit for treating HSCT-TMA in a human pediatric patient comprises: (a) a dose of an anti-C5 antibody or antigen binding fragment thereof, comprising CDR1, CDR2 and CDR3 domains of the heavy chain variable region having the sequence set forth in SEQ ID NO:12, and CDR1, CDR2 and CDR3 domains of the light chain variable region having the sequence set forth in SEQ ID NO:8; and (b) instructions for using the anti-C5 antibody or antigen binding fragment thereof, according to any of the methods described herein.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 300 mg; and
(e) a supplemental dose of 300 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 400 mg; and
(e) a supplemental dose of 400 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 600 mg, and
(e) a supplemental dose of 600 mg, with instructions that the supplemental dose is administered to a patient who receives RBC transfusion within 2 weeks of a maintenance dose, wherein the supplemental dose is administered two weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 800 mg, and
(e) a supplemental dose of 800 mg, with instructions that the supplemental dose is administered to a patient who receives RBC transfusion within 2 weeks of a maintenance dose, wherein the supplemental dose is administered two weeks after a maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing 20 to <30 kg:
(a) once on Day 1 at a loading dose of 900 mg;
(b) once on Day 5 at a loading dose of 300 mg;
(c) once on Day 10 at a loading dose of 300 mg;
(d) on Day 15 and every eight weeks thereafter at a maintenance dose of 2100 mg; and
(e) a supplemental dose of 2100 mg, with instructions that the supplemental dose is administered to a patient who receives RBC transfusion within 4 weeks of a maintenance dose, wherein the supplemental dose is administered four weeks after the maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg;
(d) on Day 15 and every eight weeks thereafter at a maintenance dose of 2700 mg, and (e) a supplemental dose of 2700 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg;
(d) on Day 15 and every eight weeks thereafter at a maintenance dose of 3000 mg, and (e) a supplemental dose of 3000 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg; and
(d) on Day 15 and every eight weeks thereafter at a maintenance dose of 3300 mg, and (e) a supplemental dose of 3300 mg, with instructions that the supplemental dose is administered to a patient who receives RBC transfusion within 4 weeks of a maintenance dose, wherein the supplemental dose is administered four weeks after that maintenance dose.

In another embodiment, the kit comprises loading, maintenance, and supplemental doses of an anti-C5 antibody, or antigen binding fragment thereof, wherein the anti-C5 antibody, or antigen binding fragment thereof, is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg; and
(d) on Day 15 and every eight weeks thereafter at a maintenance dose of 3600 mg, and (e) a supplemental dose of 3600 mg, with instructions that the supplemental dose is administered to a patient who receives RBC transfusion within 4 weeks of a maintenance dose, wherein the supplemental dose is administered four weeks after that maintenance dose.

The following examples are merely illustrative and should not be construed as limiting the scope of this disclosure in any way as many variations and equivalents will become apparent to those skilled in the art upon reading the present disclosure. The contents of all references, GENBANK entries, patents and published patent applications cited throughout this application are expressly incorporated herein by reference.

### EXAMPLES

### Example 1: Ravulizumab Dose Confirmation Analysis in Global Phase 3 Study to Assess Pharmacokinetics/Pharmacodynamics in Adults and Adolescents Who Developed Thrombotic Microangiopathy After Hematopoietic Stem Cell Transplant

A pharmacokinetic-pharmacodynamic (PK-PD) analysis was conducted to confirm the ravulizumab dose regimen being used to treat patients with HSCT-TMA in a phase 3 study, which is described in WO/2022/036151, the contents of which are expressly incorporated herein by reference. In addition, a novel supplemental dosing regimen was implemented in the present study for patients who received a platelet or RBC transfusion during treatment.

Stage 1 of the phase 3 study of ravulizumab for the treatment of adults and adolescents (aged ≥12 years) with HSCT-TMA (NCT04543591) was an open-label, single-arm period designed to confirm the ravulizumab dose regimen. Participants received weight-based loading doses on Days 1, 5 and 10, followed by a maintenance dose on Day 15 and every 8 weeks thereafter. A supplemental dose was given within 24 hours after any platelet or red blood cell (RBC) transfusion. There was no limit on the number of supplemental doses. Blood samples for analysis were taken during the treatment period. Dose confirmation analysis (DCA) was performed once at least the first 10 patients enrolled had completed Day 21.

DCA involved three steps. Step 1: PK and free C5 (primary PD) data were visualized. Previously established thresholds indicating complete C5 inhibition were used to empirically confirm that the dose regimen resulted in appropriate ravulizumab concentrations and was providing immediate, complete and sustained inhibition of free C5. Step 2: population pharmacokinetic (popPK) modeling of ravulizumab concentration-time data. Body weight and transfusions were applied to a previously developed aHUS popPK model to individualize the predicted exposure for each patient with HSCT-TMA and to assess the impact of these factors from a patient population level. Standard assessments were used for model qualification. Step 3: the qualified popPK model was used to perform simulations confirming/adjusting the ravulizumab dose regimen, including transfusion-related supplemental doses. Monte Carlo simulation of concentration-time profiles was performed to assess if randomly generated exposures (based on population variability of PK parameters) would remain above the PK threshold, thus ensuring complete terminal complement inhibition.

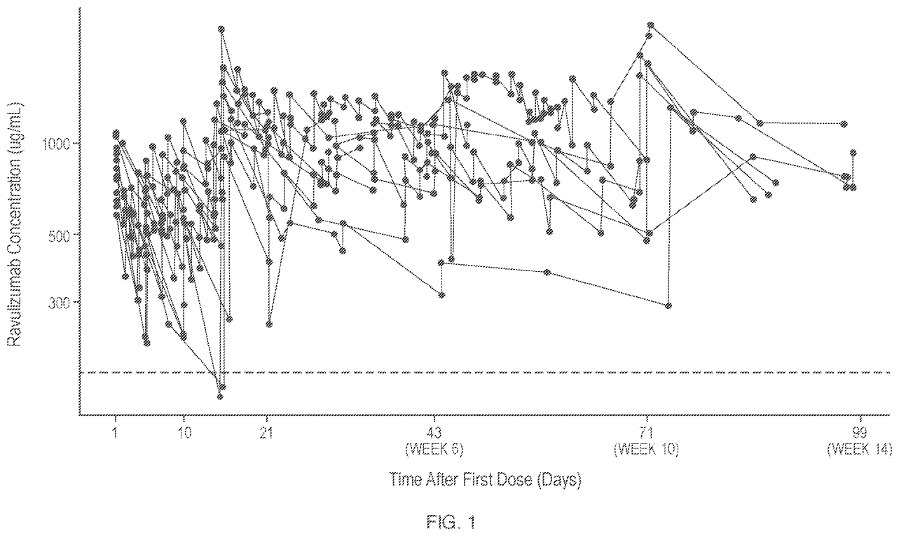

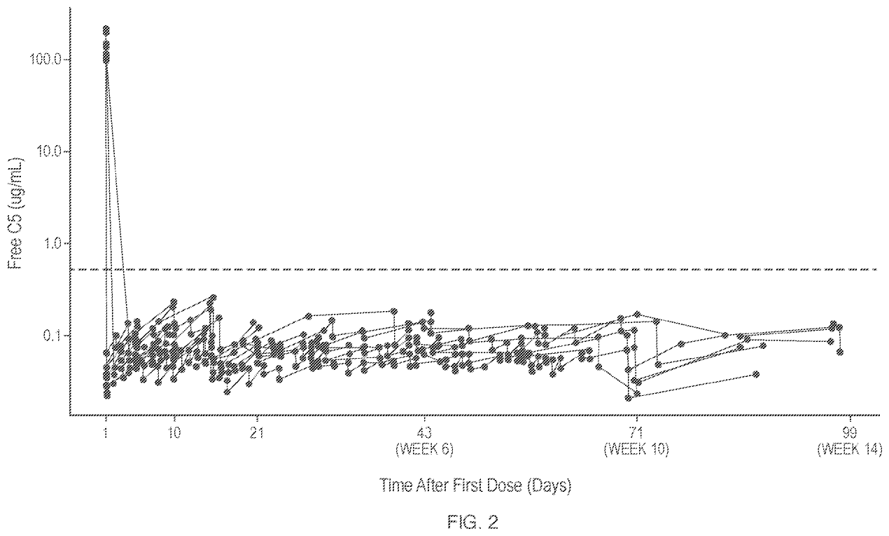

A total of 361 PK observations from 14 patients were included in the empiric evaluation (FIGS. 1 and 2), revealing that the PK and PD thresholds were both met using the protocol dose regimens. In addition, FIG. 2 shows that the Day 5 and Day 10 loading doses appropriately managed high baseline levels of complement complexes, with ravulizumab concentrations maintained at >175 μg/mL. The qualified popPK model characterized the dose-concentration relationship well. Further, it was determined that faster clearance occurred following RBC transfusions. Taking this into account, the simulations focused on adjusting/optimizing the transfusion-related supplemental dosing of ravulizumab.

The simulation confirmed that the supplemental dose regimen could be updated such that patients with body weight≥30 kg who received a RBC transfusion in the 4 weeks following a maintenance dose were administered a supplemental dose of ravulizumab 4 weeks after that maintenance dose. Dose simulation predicted the mean (standard deviation) minimum and maximum concentrations during a dosing interval to be 474 (200) μg/mL and 1350 (430) μg/mL, respectively. The updated dose regimen is summarized in Table 1. The Post-Transfusion Supplemental Dosing Schedule is set forth in Table 2. A summary of the actions to take if one or more RBC transfusion occurs is set forth in Table 3.

TABLE 1
Updated Ravulizumab Weight-Based Dose Regimen

| | | | | Maintenance | |
|---|---|---|---|---|---|
| | Loading phase doses | | | doses | Supple- |
| Body | Day | Day | Day | (starting | mental |
| weight^{a} | 1 | 5 | 10 | Day 15) | dose^{b} |
| 30 to <40 kg | 1200 mg | 300 mg | 300 mg | 2700 mg Q8W | 2700 mg |
| 40 to <60 kg | 2400 mg | 600 mg | 600 mg | 3000 mg Q8W | 3000 mg |
| 60 to <100 | 2700 mg | 900 mg | 900 mg | 3300 mg Q8W | 3300 mg |
| kg | | | | | |
| ≥100 kg | 3000 mg | 900 mg | 900 mg | 3600 mg Q8W | 3600 mg |

^{a}Weight-based dosing is based on the patient's body weight recorded at the day of the infusion visit. If the weight at the day of the infusion cannot be obtained, the weight recorded during the previous study visit was used.
^{b}Participants with body weight ≥30 kg who received any red blood cell transfusion in the 4 weeks following a maintenance dose were administered a supplemental dose of ravulizumab 4 weeks following that maintenance dose. Supplemental dosing is not required during the loading phase and is not required following platelet transfusion. Q8W, every 8 weeks.

TABLE 2
Post-Transfusion Supplemental Dosing Schedule

| Regular Protocol Visit | | | | | | | | | | | | | |
|---|---|---|---|---|---|---|---|---|---|---|---|---|---|
| V1 | V2 | V3 | V4 | V5 | V6 | V7 | V8 | V9 | V10 | V11 | V12 | V13 | V14 |
| Days | | | | | | | | | | | | | |
| D | D | D | D | D | D | D | D | D | D | D | D | D | D |
| 1 | 5 | 10 | 15 | 21 | 29 | 43 | 57 | 71 | 85 | 99 | 127 | 155 | 183 |

| | | | | | | | | | |
|---|---|---|---|---|---|---|---|---|---|
| Ravulizumab/ | X | X | X | X | | X | | X | |
| Placebo | | | | | | | | | |
| regular dosing | | | | | | | | | |
| regimen | | | | | | | | | |
| (loading + | | | | | | | | | |
| maintenance) | | | | | | | | | |
| Post- | | | | | X | | X | | X |
| transfusion | | | | | | | | | |
| supplemental | | | | | | | | | |
| dosing is | | | | | | | | | |
| required based | | | | | | | | | |
| on when the | | | | | | | | | |
| patient | | | | | | | | | |
| received RBC | | | | | | | | | |
| transfusion. | | | | | | | | | |

TABLE 3
Action to Take if One or More RBC Transfusions Occur

| If one or more RBC | |
|---|---|
| transfusion occurs: | Action to take: |
| Between D 15 (after | A post-transfusion supplemental dose is to |
| maintenance dose) and D 43 | be given at D 43 |
| Between D 44 and D 71 | No post-transfusion supplemental dose is |
| | required until the next maintenance dose |
| | (D 71) |
| Between D 71 (post | A post-transfusion supplemental dose is to |
| maintenance dose) and D 99 | be given at D 99 |
| Between D 100 and D 127 | No post-transfusion supplemental dose is |
| | required until the next maintenance dose |
| | (D 127) |
| Between D 127 (post | A post-transfusion supplemental dose is to |
| maintenance dose) and D 155 | be given at D 155 |
| After D 155 | No post-transfusion supplemental dose is |
| | required until the end of the treatment |
| | period |

This thorough DCA confirmed that the updated supplemental ravulizumab dose regimen achieved immediate, complete and sustained terminal complement inhibition. The dose regimen has thus been optimized for use in Stage 2 (double-blind, randomized, placebo-controlled period) of the phase 3 study of ravulizumab efficacy and safety in HSCT-TMA.

### Example 2: Dosing Regimen for Patients Weighing Less Than 30 kg

A PK-PD analysis was conducted to confirm the ravulizumab dose regimen being used to treat patients with HSCT-TMA in a phase 3 study, substantially as described above in Example 1 and WO/2022/036151. A supplemental dosing regimen was implemented in the present study for patients weighing less than 30 kg who received a platelet or RBC transfusion during treatment. The Post-Transfusion Supplemental Dosing Schedule is set forth in Table 4.

TABLE 4
Post-Transfusion Supplemental Dosing Schedule
for Patients Weighing Less Than 30 kg

| | | | | Maintenance | |
|---|---|---|---|---|---|
| | Loading Phase Doses | | | Doses | Supple- |
| | Day | Day | Day | Starting | mental |
| Weight | 1 | 5 | 10 | Day 15 | Dose |
| 5 to <10 kg | 600 mg | 300 mg | 300 mg | 300 mg | 300 mg two |
| | | | | q4w | weeks post MD |
| 10 to <20 kg | 600 mg | 300 mg | 300 mg | 600 mg | 600 mg two |
| | | | | q4w | weeks post MD |
| 20 to <30 kg | 900 mg | 300 mg | 300 mg | 2100 mg | 2100 mg four |
| | | | | q8w | weeks post MD |

Ten patients were evaluated for possible dose confirmation analysis (DCA). One of the ten patients was unevaluable. Two of the patients had high frequency RBC transfusions (referred to as “high ravulizumab clearance”). Seven patients had PK>175 mcg/mL during treatment (referred to as “low frequency RBC transfusions”).

The initial DCA for 9/9 patients failed (<175 mcg/mL). However, upon further investigation, it was determined that the “high ravulizumab clearance” patients were responsible for the failed DCA. Upon removal of these two dose unconfirmed patients (22%) the DCA confirmed dose for the remaining 7/9 patients. However, patients <30 kg could benefit further from a revised dose regimen because it is not possible to know when a patient may become a “high ravulizumab clearance” patient.

### Example 3: Revised Dosing Regimen for Patients Weighing Less Than 30 kg

In Example 2, the dose regimen set forth in Table 4 was confirmed for 7/9 patients (22%). However, it was determined that certain patients weighing <30 kg with frequent RBC transfusions (e.g., high ravulizumab clearance” patients weighing <30 kg) may not maintain target trough level (175 mcg/mL) and therefore, not achieve complete C5 inhibition. Therefore, this particular group of patients could benefit further from a revised dose regimen. Accordingly, a revised dosing regimen as set forth in Table 5 is assessed in this patient group, substantially according to the methods described in Examples 1-2 and WO/2022/036151, with the noted modifications.

TABLE 5
Revised Dosing Schedule for Patients Weighing Less Than 30 kg

| | Loading Phase Doses | | | Maintenance Doses | Supplemental |
|---|---|---|---|---|---|
| Weight | Day 1 | Day 5 | Day 10 | Starting Day 15 | Dose |
| 5 to <10 kg | 600 mg | 300 mg | 300 mg | 400 mg q4w | 400 mg two weeks post MD |
| 10 to <20 kg | 600 mg | 300 mg | 300 mg | 800 mg q4w | 800 mg two weeks post MD |
| 20 to <30 kg | 900 mg | 300 mg | 300 mg | 2100 mg q8w | 2100 mg four weeks post MD |
| | | | | (no change compared | (no change compared |
| | | | | to dose in Example 2) | to dose in Example 2) |

For all patients, the need for a post-RBC transfusion supplemental dose of ravulizumab is assessed when the patient reaches the mid-way point between maintenance doses as set forth in Table 6. For participants with body weight ≥30 kg, a post-RBC transfusion supplemental dose is administered if the participant has received at least 1 RBC transfusion since the last maintenance dose. For participants with body weight <20 kg or with body weight≥20 to <30 kg, a clinical algorithm is followed to assess the need for post-RBC transfusion supplemental dosing

TABLE 6
Timing of Post-RBC Transfusion Supplemental Dosing

| | Timing of Assessment for Post-RBC Transfusion |
|---|---|
| Weight | Supplemental Dose |
| <20 kg | 2 weeks (±3 days) after each maintenance dose |
| | (i.e., Days 29, 57, 85, 113, 141, and 169).* |
| ≥20 to | 4 weeks (±3 days) after each maintenance dose |
| <30 kg | (i.e., Days 43, 99, and 155).* |
| ≥30 kg | 4 weeks (±3 days) after each maintenance dose |
| | (i.e., Days 43, 99, and 155).* |

*The supplemental dose is administered within the same window for each weight categories. If supplemental dosing is required at the same time as a scheduled study visit, the dosing can take place at that visit.

The revised dosing regimen triggers a clinical algorithm when RBC transfusion day frequency exceeds >3.5 days/week (i.e., the threshold for when >10% patients have trough level <175 mcg/mL). Specifically, the clinical algorithm monitors the number of RBC transfusion days (the number of days with at least 1 RBC transfusion) and clinical worsening. Previously, the investigator assessed only whether the participant has received at least one RBC transfusion since the last maintenance dose. The revised dosing regimen is proposed to increase both maintenance and supplemental doses to mitigate issues. The clinical algorithm is set forth in FIG. 3 and summarized in Table 7.

TABLE 7
Assessment of the Need for Post-RBC Transfusion Supplemental Dosing

| | Clinical Algorithm for Assessment of Post-RBC Transfusion | |
|---|---|---|
| Weight | Supplemental Dosing | |
| <20 kg | 1. | Has the participant received any RBC transfusions since the last |
| | | maintenance dose? |
| | | If yes, proceed to question #2 |
| | | If no, await next scheduled maintenance dose |
| | 2. | Has the participant received RBC transfusions on >7 days since the last |
| | | maintenance dose? |
| | | If yes, proceed to question #3 |
| | | If no, administer supplemental dose |
| | 3. | Does the participant meet criteria for clinical worsening? |
| | | If yes, the Investigator must contact the Medical Monitor to determine |
| | | if the participant should continue study treatment and receive a |
| | | supplemental dose |
| | | If no, proceed to question #4 |
| | 4. | In the Investigator's clinical judgement, is the participant benefitting from |
| | | study treatment? |
| | | If yes, administer supplemental dose |
| | | If no, Investigator must determine if study treatment discontinuation |
| | | is needed |
| ≥20 to <30 | 1. | Has the participant received any RBC transfusions since the last |
| kg | | maintenance dose? |
| | | If yes, proceed to question #2 |
| | | If no, await next scheduled maintenance dose |
| | 2. | Has the participant received RBC transfusions on >14 days since the last |
| | | maintenance dose? |
| | | If yes, proceed to question #3 |
| | | If no, administer supplemental dose |
| | 3. | Does the participant meet criteria for clinical worsening? |
| | | If yes, contact the Medical Monitor to determine if the participant |
| | | should continue study treatment and receive a supplemental dose |
| | | If no, proceed to question #4 |
| | 4. | In the Investigator's clinical judgement is the participant benefitting from |
| | | study treatment? |
| | | If yes, administer supplemental dose |
| | | If no, Investigator must determine if study treatment discontinuation |
| | | is needed |
| ≥30 kg | 1. | Has the participant received any RBC transfusions since the last |
| | | maintenance dose? |
| | | If yes, administer supplemental dose |
| | | If no, await next scheduled maintenance dose |

For all participants, if no RBC transfusions have been administered, supplemental dosing is not required.

Supplemental doses of ravulizumab are allowed during the Treatment Period following administration of the first Maintenance Dose on Day 15 for patients who demonstrate clinical worsening. Clinical worsening is defined as meeting 2 or more of the following criteria:
1. Doubling of serum creatinine from baseline or new requirement of dialysis;
2. Increase in LDH>25% compared to baseline;
3. Reduction in platelets of >25% compared to baseline;
4. Reduction in hemoglobin >10% compared to baseline; and/or
5. Increased requirement for platelets or RBCs at any time.

The criteria for clinical worsening is confirmed by two independent samples collected at least 8 hours apart (ideally 24 hours apart).

The supplemental dosing regimen according the present disclosure is summarized in Table 8, below:

TABLE 8
Supplemental Dosing Regimen

| | Loading Phase Dosages | | | Maintenance | |
|---|---|---|---|---|---|
| Weight | Day 1 | Day 5 | Day 10 | Dose at day 15 | Supplemental Dose |
| 5 to <10 kg | 600 mg | 300 mg | 300 mg | 400 mg q4w | 400 mg 2 wks post MD |
| 10 < 20 kg | 600 mg | 300 mg | 300 mg | 800 mg q4w | 800 mg 2 wks post MD |
| 20 < 30 kg | 900 mg | 300 mg | 300 mg | 2100 mg q8w | 2100 mg 4 wks post MD |
| 30 < 40 kg | 1200 mg | 300 mg | 300 mg | 2700 mg q8w | 2700 mg 4 wks post MD |
| 40 < 60 kg | 2400 mg | 600 mg | 600 mg | 3000 mg q8w | 3000 mg 4 wks post MD |
| 60 < 100 kg | 2700 mg | 900 mg | 900 mg | 3300 mg q8w | 3300 mg 4 wks post MD |
| ≥100 kg | 3000 mg | 900 mg | 900 mg | 3600 mg q8w | 3600 mg 4 wks post MD |

The minimum assessments to be performed during any supplemental dosing visit in which ravulizumab is administered include a urine pregnancy test, predose PK/PD blood sample collection, and vital signs.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SS
SEQ ID NO: 8
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTFGQ
GTKVEIK
SEQ ID NO: 9
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVMHEA LHNHYTQKSL SLSLGK
SEQ ID NO: 10
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY
TLPPSQEEMT NQVSLTCLVK GFYPSDIAVE WESNGQPENN YKTTPPVLDS
DGSFFLYSRL TVDKSRWQEG NVFSCSVMHE ALHNHYTQKS LSLSLGK
SEQ ID NO: 11
DIQMTQSPSS LSASVGDRVT ITCGASENIY GALNWYQQKP GKAPKLLIYG
ATNLADGVPS RFSGSGSGTD FTLTISSLQP EDFATYYCQN VLNTPLTEGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSEN RGEC
SEQ ID NO: 12
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SS
SEQ ID NO: 13
ASTKGPSVFP LAPCSRSTSE STAALGCLVK DYFPEPVTVS WNSGALTSGV
HTFPAVLQSS GLYSLSSVVT VPSSNFGTQT YTCNVDHKPS NTKVDKTVER
KCCVECPPCP APPVAGPSVF LFPPKPKDTL MISRTPEVTC VVVDVSQEDP
EVQFNWYVDG VEVHNAKTKP REEQFNSTYR VVSVLTVLHQ DWLNGKEYKC
KVSNKGLPSS IEKTISKAKG QPREPQVYTL PPSQEEMTKN QVSLTCLVKG
FYPSDIAVEW ESNGQPENNY KTTPPVLDSD GSFFLYSRLT VDKSRWQEGN
VFSCSVLHEA LHSHYTQKSL SLSLGK
SEQ ID NO: 14
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVLH EALHSHYTQK SLSLSLGK
SEQ ID NO: 15
ASTKGPSVFP LAPCSRSTSE STAALGCLVK DYFPEPVTVS WNSGALTSGV
HTFPAVLQSS GLYSLSSVVT VTSSNFGTQT YTCNVDHKPS NTKVDKTVER
KCCVECPPCP APPVAGPSVF LFPPKPKDTL YITREPEVTC VVVDVSHEDP
EVQFNWYVDG MEVHNAKTKP REEQFNSTER VVSVLTVVHQ DWLNGKEYKC
KVSNKGLPAP IEKTISKTKG QPREPQVYTL PPSREEMTKN QVSLTCLVKG
FYPSDIAVEW ESNGQPENNY KTTPPMLDSD GSFFLYSKLT VDKSRWQQGN
VFSCSVMHEA LHNHYTQKSL SLSPGK
SEQ ID NO: 16
QVQLVQSGAE VKKPGASVKV SCKASGYIFS NYWIQWVRQA PGQGLEWMGE
ILPGSGSTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVTSSNEGT
QTYTCNVDHK PSNTKVDKTV ERKCCVECPP CPAPPVAGPS VFLFPPKPKD
TLYITREPEV TCVVVDVSHE DPEVQFNWYV DGMEVHNAKT KPREEQFNST
FRVVSVLTVV HQDWLNGKEY KCKVSNKGLP APIEKTISKT KGQPREPQVY
TLPPSREEMT KNQVSLTCLV KGFYPSDIAV EWESNGQPEN NYKTTPPMLD
SDGSFFLYSK LTVDKSRWQQ GNVFSCSVMH EALHNHYTQK SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAE VKKPGASVKV SCKASGHIFS NYWIQWVRQA PGQGLEWMGE
ILPGSGHTEY TENFKDRVTM TRDTSTSTVY MELSSLRSED TAVYYCARYF
FGSSPNWYFD VWGQGTLVTV SSASTKGPSV FPLAPCSRST SESTAALGCL
VKDYFPEPVT VSWNSGALTS GVHTFPAVLQ SSGLYSLSSV VTVPSSNEGT
QTYTCNVDHK PSNTKVDKTV ERKCCVECPP CPAPPVAGPS VFLFPPKPKD
TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY
TLPPSQEEMT KNQVSLTCLV KGFYPSDIAV EWESNGQPEN NYKTTPPVLD
SDGSFFLYSR LTVDKSRWQE GNVFSCSVMH EALHNHYTQK SLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAE VKKPGSSVKV SCKASGGTFS SYAISVWRQA PGQGLEWMGG
IGPFFGTANY AQKFQGRVTI TADESTSTAY MELSSLRSED TAVYYCARDT
PYFDYWGQGT LVTVSS
SEQ ID NO: 28
DIELTQPPSV SVAPGQTARI SCSGDSIPNY YVYWYQQKPG QAPVLVIYDD
SNRPSGIPER FSGSNSGNTA TLTISGTQAE DEADYYCQSF DSSLNAEVFG
GGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAE VKKPGESLKI SCKGSGYSFT NYISWVRQMP GKGLEWMGII
DPDDSYTEYS PSFQGQVTIS ADKSISTAYL QWSSLKASDT AMYYCARYEY
GGFDIWGQGT LVTVSS
SEQ ID NO: 36
SYELTQPPSV SVAPGQTARI SCSGDNIGNS YVHWYQQKPG QAPVLVIYKD
NDRPSGIPER FSGSNSGNTA TLTISGTQAE DEADYYCGTY DIESYVFGGG
TKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGG LVQPGGSLRL SCAASGFTSH SSYYVAWVRQ APGKGLEWVG
AIYTGSGATY KASWAKGRFT ISKDTSKNQV VLTMTNMDPV DTATYYCASD
GGYDYPTHAM HYWGQGTLVT VSS
SEQ ID NO: 44
DVVMTQSPSS LSASVGDRVT ITCQASQNIG SSLAWYQQKP GQAPRLLIYG
ASKTHSGVPS RFSGSGSGTD FTLTISSLQP EDVATYYCQS TKVGSSYGNH
FGGGTKVEIK
SEQ ID NO: 45
QVQLVESGGG LVQPGRSLRL SCAASGFTVH SSYYMAWVRQ APGKGLEWVG
AIFTGSGAEY KAEWAKGRVT ISKDTSKNQV VLTMTNMDPV DTATYYCASD
AGYDYPTHAM HYWGQGTLVT VSSASTKGPS VFPLAPSSKS TSGGTAALGC
LVKDYFPEPV TVSWNSGALT SGVHTFPAVL QSSGLYSLSS VVTVPSSSLG
TQTYICNVNH KPSNTKVDKK VEPKSCDKTH TCPPCPAPEL RRGPKVFLFP
PKPKDTLMIS RTPEVTCVVV DVSHEDPEVK FNWYVDGVEV HNAKTKPREE
QYNSTYRVVS VLTVLHQDWL NGKEYKCKVS NKGLPSSIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVLHEALHA HYTRKELSLS
P
SEQ ID NO: 46
DIQMTQSPSS LSASVGDRVT ITCRASQGIS SSLAWYQQKP GKAPKLLIYG
ASETESGVPS RFSGSGSGTD FTLTISSLQP EDFATYYCQN TKVGSSYGNT
FGGGTKVEIK RTVAAPSVFI FPPSDEQLKS GTASVVCLLN NFYPREAKVQ
WKVDNALQSG NSQESVTEQD SKDSTYSLSS TLTLSKADYE KHKVYACEVT
HQGLSSPVTK SFNRGEC
SEQ ID NO: 47
QVQLQESGPGLVKPSETLSLTCTVSGDSVSSSYWTWIRQPPGKGLEWIGYIYYS
GSSNYNPSLKSRATISVDTSKNQFSLKLSSVTAADTAVYYCAREGNVDTTMIFD
YWGQGTLV TVSS
SEQ ID NO: 48
AIQMTQSPSSLSASVGDRVTITCRASQGIRNDLGWYQQKPGKAPKLLIYAASSL
QSGVPSRFAGRGSGTDFTLTISSLQPEDFATYYCLQDENYPWTFGQGTKVEIK
SEQ ID NO: 49
QVQLQESGPGLVKPSETLSLTCTVSGDSVSSSYWTWIRQPPGKGLEWIGYIYYS
GSSNYNPSLKSRATISVDTSKNQFSLKLSSVTAADTAVYYCAREGNVDTTMIFD
YWGQGTLVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEAL
HNHYTQKSLSLSLGK
SEQ ID NO: 50
AIQMTQSPSSLSASVGDRVTITCRASQGIRNDLGWYQQKPGKAPKLLIYAASSL
QSGVPSRFAGRGSGTDFTLTISSLQPEDFATYYCLQDENYPWTFGQGTKVEIKR
TVAAPSVFIEPPSDEQLKSGTASVVCLLNNFYPREAKVQWKVDNALQSGNSQES
VTEQDSKDSTYSLSSTLTLSKADYEKHKVYACEVTHQGLSSPVTKSENRGEC

What is claimed is:

1. A method of treating a human patient with hematopoietic stem cell transplant-associated thrombotic microangiopathy (HSCT-TMA), the method comprising administering to the patient an effective amount of an anti-C5 antibody, or antigen binding fragment thereof, comprising CDR1, CDR2 and CDR3 heavy chain sequences as set forth in SEQ ID NOs: 19, 18 and 3, respectively, and CDR1, CDR2 and CDR3 light chain sequences as set forth in SEQ ID NOs: 4, 5 and 6, respectively, wherein the anti-C5 antibody or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
(i) every four weeks thereafter at a maintenance dose of: 300 or 400 mg to a patient weighing 5 to <10 kg or 600 or 800 mg to a patient weighing 10 to <20 kg, or
(ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
(i) to a patient weighing <30 kg who receives a red blood cell (RBC) transfusion within a specified timeframe of a maintenance dose, wherein the supplemental dose is administered at the specified timeframe following the maintenance dose, at a dose of:
a. 300 or 400 mg to a patient weighing 5 to <10 kg, wherein the specified timeframe is two weeks,
b. 600 or 800 mg to a patient weighing 10 to <20 kg, wherein the specified timeframe is two weeks, or
c. 2100 mg to a patient weighing 20 to <30 kg, wherein the specified timeframe is four weeks, or
(ii) to a patient weighing ≥30 kg who receives a red blood cell (RBC) transfusion within four weeks of a maintenance dose, wherein the supplemental dose is administered four weeks after that maintenance dose at a dose of:
a. 2700 mg to a patient weighing ≥30 to <40 kg,
b. 3000 mg to a patient weighing ≥40 to <60 kg,
c. 3300 mg to a patient weighing ≥60 to <100 kg, or
d. 3600 mg to a patient weighing ≥100 kg.

2. The method of claim 1, wherein the anti-C5 antibody, or antigen binding fragment thereof, further comprises a variant human Fc constant region that binds to human neonatal Fc receptor (FcRn), wherein the variant human Fc constant region comprises Met429Leu and Asn435Ser substitutions at residues corresponding to methionine 428 and asparagine 434 of a native human IgG Fc constant region, each in EU numbering.

3. The method of claim 1 or 2, wherein the anti-C5 antibody comprises a heavy chain variable region set forth in SEQ ID NO:12 and a light chain variable region set forth in SEQ ID NO:8.

4. The method of any one of the preceding claims, wherein the anti-C5 antibody further comprises a heavy chain constant region set forth in SEQ ID NO:13.

5. The method of any one of the preceding claims, wherein the antibody comprises a heavy chain polypeptide comprising the amino acid sequence set forth in SEQ ID NO: 14 and a light chain polypeptide comprising the amino acid sequence set forth in SEQ ID NO:11.

6. The method of any one of the preceding claims, wherein the anti-C5 antibody binds to human C5 at pH 7.4 and 25 C with an affinity dissociation constant (K_{D}) that is in the range 0.1 nM≤K_{D}≤1 nM (e.g., about 0.5 nM).

7. The method of any one of the preceding claims, wherein the anti-C5 antibody binds to human C5 at pH 6.0 and 25 C with a K_{D}≥10 nM (e.g., about 22 nM).

8. The method of any one of the preceding claims, wherein the anti-C5 antibody is administered to a patient weighing 5 to <10 kg:
(a) once on Day 1 at a loading dose of 600 mg;
(b) once on Day 5 at a loading dose of 300 mg;
20 (c) once on Day 10 at a loading dose of 300 mg;
(d) on Day 15 and every four weeks thereafter at a maintenance dose of 300 mg; and
(e) a supplemental dose of 300 mg, to a patient who receives RBC transfusion within 2 weeks of a maintenance dose, and wherein the supplemental dose is administered two weeks after the maintenance dose.

9. The method of any one of claims 1-7, wherein the anti-C5 antibody is administered to a patient weighing 5 to <10 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 400 mg; and
(e) a supplemental dose of 400 mg, to a patient who receives RBC transfusion within 2 weeks of a maintenance dose, and wherein the supplemental dose is administered two weeks after the maintenance dose.

10. The method of any one of claims 1-7, wherein the anti-C5 antibody is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 600 mg, and
(e) a supplemental dose of 600 mg, to a patient who receives RBC transfusion within 2 weeks of a maintenance dose, and wherein the supplemental dose is administered two weeks after the maintenance dose.

11. The method of any one of claims 1-7, wherein the anti-C5 antibody is administered to a patient weighing 10 to <20 kg:
(a) once on Day 1 at a loading dose of 600 mg;
(b) once on Day 5 at a loading dose of 300 mg;
(c) once on Day 10 at a loading dose of 300 mg;
(d) on Day 15 and every four weeks thereafter at a maintenance dose of 800 mg, and
(e) a supplemental dose of 800 mg, to a patient who receives RBC transfusion within 2 weeks of a maintenance dose, and wherein the supplemental dose is administered two weeks after the maintenance dose.

12. The method of any one of claims 1-7, wherein the anti-C5 antibody is administered to a patient weighing 20 to <30 kg:
(a) once on Day 1 at a loading dose of 900 mg;
(b) once on Day 5 at a loading dose of 300 mg;
(c) once on Day 10 at a loading dose of 300 mg;
(d) on Day 15 and every eight weeks thereafter at a maintenance dose of 2100 mg; and
(e) a supplemental dose of 2100 mg, to a patient who receives RBC transfusion within 4 weeks of a maintenance dose, and wherein the supplemental dose is administered four weeks after the maintenance dose.

13. The method of any one of claims 8-12, wherein the supplemental dose is administered if warranted per a clinical algorithm as set forth in FIG. 3.

14. The method of any one of claims 1-7, wherein the anti-C5 antibody is administered to a patient weighing ≥30 to <40 kg:
(a) once on Day 1 at a loading dose of 1200 mg;
(b) once on Day 5 at a loading dose of 300 mg;
(c) once on Day 10 at a loading dose of 300 mg;
(d) on Day 15 and every eight weeks thereafter at a maintenance dose of 2700 mg.

15. The method of claim 14, wherein the patient receives a red blood cell (RBC) transfusion within four weeks of a maintenance dose and is then administered a supplemental dose of 2700 mg four weeks after that maintenance dose.

16. The method of any one of claims 1-7, wherein the anti-C5 antibody is administered to a patient weighing ≥40 to <60 kg:
(a) once on Day 1 at a loading dose of 2400 mg;
(b) once on Day 5 at a loading dose of 600 mg;
(c) once on Day 10 at a loading dose of 600 mg; and
(d) on Day 15 and every eight weeks thereafter at a maintenance dose of 3000 mg.

17. The method of claim 16, wherein the patient receives a red blood cell (RBC) transfusion within four weeks of a maintenance dose and is then administered a supplemental dose of 3000 mg four weeks after that maintenance dose.

18. The method of any one of claims 1-7, wherein the anti-C5 antibody is administered to a patient weighing ≥60 to <100 kg:
(a) once on Day 1 at a loading dose of 2700 mg;
(b) once on Day 5 at a loading dose of 900 mg;
(c) once on Day 10 at a loading dose of 900 mg; and
(d) on Day 15 and every eight weeks thereafter at a maintenance dose of 3300 mg.

19. The method of claim 18, wherein the patient receives a red blood cell (RBC) transfusion within four weeks of a maintenance dose and is then administered a supplemental dose of 3300 mg four weeks after that maintenance dose.

20. The method of any one of claims 1-7, wherein the anti-C5 antibody is administered to a patient weighing ≥100 kg:
(a) once on Day 1 at a loading dose of 3000 mg;
(b) once on Day 5 at a loading dose of 900 mg;
(c) once on Day 10 at a loading dose of 900 mg; and
(d) on Day 15 and every eight weeks thereafter at a maintenance dose of 3600 mg.

21. The method of claim 20, wherein the patient receives a red blood cell (RBC) transfusion within four weeks of a maintenance dose and is then administered a supplemental dose of 3600 mg four weeks after that maintenance dose.

22. The method of any one of the preceding claims, wherein the treatment maintains a serum trough concentration of the anti-C5 antibody of 175 μg/mL or greater.

23. The method of any one of the preceding claims, wherein the treatment maintains a serum trough concentration of the anti-C5 antibody of 200 μg/mL or greater.

24. The method of any one of the preceding claims, wherein the anti-C5 antibody is formulated for intravenous administration.

25. The method of any one of the preceding claims, wherein the treatment is an administration cycle comprising a total of 26 weeks of treatment.

26. The method of any one of the preceding claims, wherein the treatment results in a reduction or cessation in microangiopathic hemolytic anemia, thrombocytopenia, endothelial injury, kidney damage, kidney failure, serositis, pulmonary hypertension, and multisystem organ failure compared to baseline.

27. The method of any one of the preceding claims, wherein the treatment results in (a) platelet count ≥50,000/mm³ without transfusion support during the prior 7 days, (b) LDH <1.5×ULN, and (c) absence of schistocytes (if there were schistocytes present at baseline).

28. The method of any one of the preceding claims, wherein the treatment results in (a) platelet count ≥50,000/mm³ without transfusion support during the prior 7 days, (b) LDH <1.5×ULN, (c) absence of schistocytes (if there were schistocytes present at baseline) and (d) at least 50% reduction of proteinuria from baseline.

29. The method of any one of the preceding claims, wherein the treatment results in results in a favorable hematological response.

30. The method of any one of the preceding claims, wherein the treatment results in normalization of LDH, resolution of need for red cell and platelet transfusions, and disappearance of schistocytes.

31. The method of any one of the preceding claims, wherein the treatment results in hemoglobin ≥8 g/dL without transfusion support.

32. The method of any one of the preceding claims, wherein the treatment results in a decrease in LDH, an increase in platelets, and/or an increase in hemoglobin compared to baseline.

33. The method of any one of the preceding claims, wherein the treatment results in normal levels of serum creatinine compared to baseline.

34. The method of any one of the preceding claims, wherein the treatment results in an improvement in TMA-associated organ dysfunction in the renal, cardiovascular, pulmonary, CNS, and/or GI systems compared to baseline.

35. The method of any one of the preceding claims, wherein the treatment results in terminal complement inhibition.

36. The method of any one of the preceding claims, wherein the treatment results in a reduction in adverse events.

37. The method of any one of the preceding claims, wherein the treatment results in a shift toward normal levels of biomarkers associated with vascular inflammation (e.g., shed tumor necrosis factor receptor 1 [TNF-R1]), endothelial damage and/or activation (e.g., thrombomodulin and shed vascular cell adhesion molecule 1 [VCAM-1]), renal injury (e.g., Cystatin C), and/or complement proteins and complement activation pathway products.

38. The method of any one of the preceding claims, wherein the treatment results in a shift toward normal levels of thrombomodulin (TM) and/or syndecan-1 (SYND1) and, optionally, complement factor Ba.

39. The method of any one of the preceding claims, wherein the treatment results in a change from baseline in quality of life as assessed via a Quality of Life Assessment.

40. The method of any one of the preceding claims, wherein the Quality of Life Assessment is a Quality of Life Inventory (PedsQL) Scale or an EQ-5D-5L questionnaire).

41. The method of any one of the preceding claims, wherein the treatments results in a reduction of lactate dehydrogenase (LDH) levels compared to baseline.

42. The method of any one of the preceding claims, wherein the treatment results in a reduction in free C5 concentration in the patient or reduction in red blood cell (RBC) hemolysis; particularly wherein the treatment results in free C5 concentration of 0.5 μg/mL or less and/or RBC hemolysis of 20% or less compared to an untreated patient.

43. The method of any one of the preceding claims, wherein the human patient is a pediatric patient or an adult patient, preferably a pediatric patient.

44. The method of any one of the preceding claims, wherein the human patient is an RBC transfused patient, preferably a patient who has received RBC transfusion during the maintenance dosing phase of treatment with the anti-C5 antibody.

45. The method of any one of the preceding claims, further comprising administering one or more best supportive care (BSC) measures.

46. The method of claim 45, wherein the one or more BSC measures is selected from the group consisting of transfusion support, corticosteroids, dialysis, and antihypertensive medications.

47. A kit for treating hematopoietic stem cell transplant-associated thrombotic microangiopathy (HSCT-TMA), in a human patient who has undergone an RBC transplant during the first half of a maintenance phase of treatment with an anti-C5 antibody or an antigen-binding fragment thereof, the kit comprising:
(a) loading, maintenance, and supplemental doses of the anti-C5 antibody, or antigen binding fragment thereof, comprising CDR1, CDR2 and CDR3 domains of the heavy chain variable region having the sequence set forth in SEQ ID NO: 12, and CDR1, CDR2 and CDR3 domains of the light chain variable region having the sequence set forth in SEQ ID NO:8; and
(b) instructions for using the anti-C5 antibody, or antigen binding fragment thereof, in the method of any one of the preceding claims.

48. An anti-C5 antibody, or antigen-binding fragment thereof, for use in treatment of an HSCT-TMA patient who has undergone RBC transfusion during a first half of a maintenance phase of treatment, wherein the anti-C5 antibody, or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
(i) every four weeks thereafter at a maintenance dose of: 300 or 400 mg to a patient weighing 5 to <10 kg or 600 or 800 mg to a patient weighing 10 to <20 kg, or
(ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
(i) to a patient weighing <30 kg who receives red blood cell (RBC) transfusion within a specified timeframe of a maintenance dose, wherein the supplemental dose is administered at the specified timeframe, at a dose of at a dose of:
a. 300 or 400 mg to a patient weighing 5 to <10 kg, wherein the specified timeframe is two weeks,
b. 600 or 800 mg to a patient weighing 10 to <20 kg, wherein the specified timeframe is two weeks, or
c. 2100 mg to a patient weighing 20 to <30 kg, wherein the specified timeframe is four weeks, or
(ii) to a patient weighing ≥30 kg who receives a red blood cell (RBC) transfusion within four weeks of a maintenance dose, wherein the supplemental dose is administered four weeks after that maintenance dose at a dose of:
a. 2700 mg to a patient weighing ≥30 to <40 kg,
b. 3000 mg to a patient weighing ≥40 to <60 kg,
c. 3300 mg to a patient weighing ≥60 to <100 kg, or
d. 3600 mg to a patient weighing ≥100 kg.

49. Use of an anti-C5, antibody or antigen-binding fragment thereof, for treatment of an HSCT-TMA patient who has undergone RBC transfusion during a first half of a maintenance phase of treatment, wherein the anti-C5 antibody, or antigen binding fragment thereof, is administered:
(a) once on Day 1 at a loading dose of: 600 mg to a patient weighing 5 to <10 kg, 600 mg to a patient weighing 10 to <20 kg, 900 mg to a patient weighing 20 to <30 kg, 1200 mg to a patient weighing ≥30 to <40 kg, 2400 mg to a patient weighing ≥40 to <60 kg, 2700 mg to a patient weighing ≥60 to <100 kg, or 3000 mg to a patient weighing ≥100 kg;
(b) once on Day 5 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg;
(c) once on Day 10 at a loading dose of: 300 mg to a patient weighing 5 to <10 kg, 300 mg to a patient weighing 10 to <20 kg, 300 mg to a patient weighing 20 to <30 kg, 300 mg to a patient weighing ≥30 to <40 kg, 600 mg to a patient weighing ≥40 to <60 kg, 900 mg to a patient weighing ≥60 to <100 kg, or 900 mg to a patient weighing ≥100 kg; and
(d) on Day 15 and
(i) every four weeks thereafter at a maintenance dose of: 300 or 400 mg to a patient weighing 5 to <10 kg or 600 or 800 mg to a patient weighing 10 to <20 kg, or
(ii) every eight weeks thereafter at a maintenance dose of: 2100 mg to a patient weighing 20 to <30 kg, 2700 mg to a patient weighing ≥30 to <40 kg, 3000 mg to a patient weighing ≥40 to <60 kg, 3300 mg to a patient weighing ≥60 to <100 kg, or 3600 mg to a patient weighing ≥100 kg;
and wherein a supplemental dose is administered:
(i) to a patient weighing <30 kg who receives red blood cell (RBC) transfusion within a specified timeframe of a maintenance dose, wherein the supplemental dose is administered at the specified timeframe, at a dose of:
a. 300 or 400 mg to a patient weighing 5 to <10 kg, wherein the specified timeframe is two weeks,
b. 600 or 800 mg to a patient weighing 10 to <20 kg, wherein the specified timeframe is two weeks, or
c. 2100 mg to a patient weighing 20 to <30 kg, wherein the specified timeframe is four weeks, or
(ii) to a patient weighing ≥30 kg who receives a red blood cell (RBC) transfusion within four weeks of a maintenance dose, wherein the supplemental dose is administered four weeks after that maintenance dose at a dose of:
a. 2700 mg to a patient weighing ≥30 to <40 kg,
b. 3000 mg to a patient weighing ≥40 to <60 kg,
c. 3300 mg to a patient weighing ≥60 to <100 kg, or
d. 3600 mg to a patient weighing ≥100 kg.

50. The anti-C5 antibody, or antigen-binding fragment thereof, of claim 48, wherein the anti-C5 antibody, or antigen binding fragment thereof, is ravulizumab (ULTOMIRIS®).

51. The use of claim 49, wherein the anti-C5 antibody, or antigen binding fragment thereof, is ravulizumab (ULTOMIRIS®).
